(19) United States
(12) Patent Application Publication
(10) Pub. No.: US 2019/0083457 A1
(43) Pub. Date: Mar. 21, 2019

(54) MITOCHONDRIAL ALDEHYDE DEHYDROGENASE-2 MODULATORS FOR PROTECTING, EXPANDING AND INCREASING THE POTENCY OF HEMATOPOIETIC STEM CELLS

(71) Applicant: The Borad of Trustees of the Leland Stanford Junior University, Stanford, CA (US)

(72) Inventors: Kenneth Weinberg, Los Altos, CA (US); Lauren D. Van Wassenhove, Menlo Park, CA (US); Daria Mochly-Rosen, Menlo Park, CA (US); Che-Hong Chen, (US)

(21) Appl. No.: 16/104,694

(22) Filed: Aug. 17, 2018

Related U.S. Application Data
(60) Provisional application No. 62/559,311, filed on Sep. 15, 2017.

(51) Int. Cl. A61K 31/36; C12N 5/0789; A61K 48/00

(52) U.S. Cl. CPC A61K 31/36; C12N 15/102; A61K 48/005; C12N 5/0647

(57) ABSTRACT

The present disclosure provides methods of protecting and expanding hematopoietic cells. The present disclosure also provides methods of increasing the potency of hematopoietic cells. Aspects of the methods include contacting a starting population of hematopoietic cells with a therapeutically effective amount of at least one ALDH2 agonist. Aspects include in vivo, in vitro and ex vivo methods of protecting, expanding and increasing the potency of hematopoietic cells. Aspects of the methods include treating an individual who is undergoing chemotherapy or radiation treatment for cancer, has been exposed to damaging toxins, has a genetic disease that leads to HSC damage, bone marrow failure, an autoimmune disease, or development of hematologic malignancies. Aspects of the methods also include treating a healthy individual who is a stem cell transplant donor.

### CROSS-REFERENCE

This application claims the benefit of U.S. Provisional Patent Application No. 62/559,311, filed Sep. 15, 2017, which application is incorporated herein by reference in its entirety.

### STATEMENT REGARDING FEDERALLY SPONSORED RESEARCH

This invention was made with government support under Grant Number AA11147 awarded by the National Institutes of Health. The government has certain rights in the invention.

### INTRODUCTION

Hematopoiesis, the formation of blood cellular components, consists of developmental cascades in which the hematopoietic stem cells (HSCs) generate lineage-committed cells and repeat the process of self-renewal. Hematopoietic stem cells are typically cells that have dual capacity for self-renewal and multilineage differentiation. The main sources of HSCs are bone marrow and umbilical cord blood. HSCs are used in transplantation applications. Bone marrow transplantation has been used in the treatment of a variety of hematological, autoimmune and malignant diseases. In conjunction with bone marrow transplantation, ex vivo hematopoietic (bone marrow) cell culture may be used to expand the population of HSCs or hematopoietic stem and progenitor cells (HSPCs). It may be desirable to purge an ex vivo hematopoietic cell culture of, for example, cancer cells with cytotoxic treatments, while preserving the viability of the HSCs or HSPCs. A variety of diseases require treatment with agents which are preferentially cytotoxic to dividing cells. Cancer cells, for example, may be targeted with cytotoxic doses of radiation or chemotherapeutic agents. A significant side-effect of this approach to cancer therapy is the pathological impact of such treatments on rapidly dividing normal cells. These normal cells may for example include hair follicles, mucosal cells and the hematopoietic cells, such as primitive bone marrow progenitor cells and stem cells. The indiscriminate destruction of HSCs and HSPCs can lead to a reduction in normal mature blood cell counts, such as leukocytes, lymphocytes and red blood cells.

HSC transplantation applications to date are somewhat limited due to the inability to amplify the HSCs ex vivo sufficiently to make the procedure viable. Some patients will not receive treatment because there are too few HSCs available to graft for successful transplant.

Accordingly, alternative agents and methods, which facilitate the preservation, expansion and potency of HSCs and HSPCs in cell cultures and in vivo are of interest.

### SUMMARY

The present disclosure provides methods of protecting and expanding hematopoietic cells. The present disclosure also provides methods of increasing the potency of hematopoietic cells. Aspects of the methods include contacting a starting population of hematopoietic cells with a therapeutically effective amount of at least one ALDH2 agonist. Aspects include in vivo, in vitro and ex vivo methods of protecting, expanding and increasing the potency of hematopoietic cells. Aspects of the methods include treating an individual who is undergoing chemotherapy or radiation treatment for cancer, has been exposed to damaging toxins, has a genetic disease that leads to HSC damage, bone marrow failure, an autoimmune disease, or development of hematologic malignancies. Aspects of the methods also include treating a healthy individual who is a stem cell transplant donor. Also disclosed herein are ex vivo methods for protecting, expanding and increasing the potency of hematopoietic cells prior to administration to an individual by either autologous or allogeneic transplant. Also disclosed herein are in vitro methods for protecting, expanding and increasing the potency of hematopoietic cells that are being genetically modified by a virus, a plasmid or CRISPR mediated gene therapy, or genomic editing. In certain embodiments, the ALDH2 agonist expands the hematopoietic cells by 2-fold or more. In certain embodiments, the ALDH2 agonist expands the hematopoietic cells by 4-fold or more. In certain embodiments, the ALDH2 agonist increases the potency of the hematopoietic cells by 2-fold or more relative to hematopoietic cells that have not been treated with the ALDH2 agonist. In certain embodiments, the ALDH2 agonist increases the potency of the hematopoietic cells by 3-fold or more relative to hematopoietic cells that have not been treated with the ALDH2 agonist.

### BRIEF DESCRIPTION OF THE DRAWINGS

FIG. 1A illustrates that treatment with an exemplary ALDH2 agonist (Alda-1) increases long-term mouse HSC.

FIG. 1B illustrates that treatment with an exemplary ALDH2 agonist (Alda-1) increases short-term HSC.

FIG. 2 illustrates that competitive repopulation shows increased repopulating ability of HSCs from Alda-1 treated mice.

### DEFINITIONS

As used herein, the term “mitochondrial aldehyde dehydrogenase-2” or “ALDH2” refers to an enzyme that oxidizes an aldehyde (e.g., a xenogenic aldehyde, a biogenic aldehyde, or an aldehyde produced from a compound that is ingested, inhaled, or absorbed) to its corresponding acid in an NAD⁺-dependent reaction. For example, ALDH2 oxidizes aldehydes derived from the breakdown of compounds, e.g., toxic compounds that are ingested, that are absorbed, that are inhaled, or that are produced during normal metabolism.

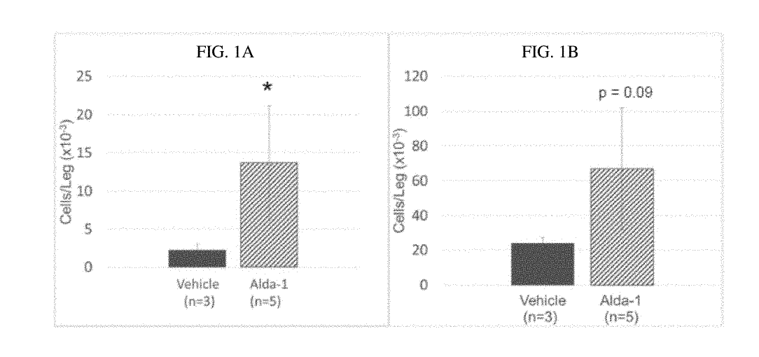

The term “ALDH2” encompasses ALDH2 from various species. Amino acid sequences of ALDH2 from various species are publicly available. For example, a human ALDH2 amino acid sequence is found under GenBank Accession Nos. AAH02967 and NP 000681; a mouse ALDH2 amino acid sequence is found under GenBank Accession No. NP 033786; and a rat ALDH2 amino acid sequence is found under GenBank Accession No. NP 115792. The term “ALDH2” as used herein also encompasses fragments, fusion proteins, and variants (e.g., variants having one or more amino acid substitutions, additions, deletions, and/or insertions) that retain ALDH2 enzymatic activity. Specific enzymatically active ALDH2 variants, fragments, fusion proteins, and the like can be verified by adapting the methods described herein. An example of an ALDH2 variant is an ALDH2 polypeptide that comprises a Glu-to-Lys substitution at amino acid position 487 of human ALDH2, as depicted in FIG. 1B (amino acid 504 of SEQ ID NO:2), or at a position corresponding to amino acid 487 of human ALDH2. This mutation is referred to as the “E487K mutation”; the “E487K variant”; or as the “Glu504Lys polymorphism”. See, e.g., Larson et al. (2005) J. Biol. Chem. 280:30550; and Li et al. (2006) J. Clin. Invest. 116:506. An ALDH2 variant retains as little as about 1% of the enzymatic activity of a corresponding wild-type ALDH2 enzyme. For example, the E487K variant retains only about 1% of the activity of an enzyme comprising the amino acid sequence depicted in FIG. 1A (SEQ ID NO:1).

The term “hematopoietic stem cells,” or “HSCs”, refers to multipotent cells capable of differentiating into all the cell types of the hematopoietic system, including, but not limited to, granulocytes, monocytes, erythrocytes, megakaryocytes, lymphocytes, dendritic cells; and self-renewal activity, i.e. the ability to divide and generate at least one daughter cell with the identical (e.g., self-renewing) characteristics of the parent cell. Bone marrow has been shown to include at least 3 multipotent populations: Long-Term (LT)-HSC, Short-Term (ST)-HSC, and Multi-Potent Progenitor (MPP, a cell population that has lost the self-renewal capacity of HSC).

The term hematopoietic stem and progenitor cells, or “HSPCs”, refers to hematopoietic stem and/or hematopoietic progenitor cells. HSPCs include hematopoietic stem cells, such as long term hematopoietic stem cells (LT-)HSCs and short term hematopoietic stem cells (ST)-HSCs, and hematopoietic progenitor cells, including multipotent progenitors (MPPs), common myeloid progenitors (CMPs), common lymphoid progenitors (CLPs), granulocyte-monocyte progenitors (GMPs) and megakaryocyte-erythroid progenitors (MEPs).

As used herein, the term “protecting” or “protection of hematopoietic cells” refers to the protection of hematopoietic cells from damaging agents, e.g., damaging toxins, chemotherapy agents, radiation treatment and the like.

As used herein, the term “expanding” or “expansion of hematopoietic cells” refers to an increase in, or expansion of, the number of hematopoietic cells relative to a starting population of hematopoietic cells.

As used herein, the terms “increasing potency” or “increasing hematopoietic cell potency” refers to a method of increasing the potency of hematopoietic cells relative to hematopoietic cells which have not been subjected to the subject methods. “Potency” refers to the differentiation potential of the cell (the potential to differentiate into different cell types). By way of example, HSCs with increased potency result in a higher number of blood cells developing following HSC transplant (HSCT). The term “damaging toxin” may refer to a poisonous substance or agent produced within living cells or organisms that is toxic to dividing cells as well as rapidly dividing normal cells. The term may also refer to damaging toxins resulting from exogenous exposures, such as exposure to environmental aldehydes.

The term “isolated compound” means a compound which has been substantially separated from, or enriched relative to, other compounds with which it occurs in nature. Isolated compounds are at least about 80%, at least about 90% pure, at least about 98% pure, or at least about 99% pure, by weight. The present invention is meant to comprehend diastereomers as well as their racemic and resolved, enantiomerically pure forms and pharmaceutically acceptable salts thereof.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient, in combination with another agent, or alone in one or more doses, to effect such treatment for the disease. The “therapeutically effective amount” will vary depending on the compound, the disease and its severity and the age, weight, etc., of the subject to be treated. The terms “subject,” “individual,” and “patient” are used interchangeably herein to a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human mammals having commercial importance (e.g., livestock and domesticated animals) are of particular interest.

“Mammal” refers to a member or members of any mammalian species, and includes, by way of example, canines; felines; equines; bovines; ovines; rodentia, etc. and primates, particularly humans. Non-human animal models, particularly mammals, e.g. a non-human primate, a murine (e.g., a mouse, a rat), lagomorpha, etc. may be used for experimental investigations.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

As used herein, a “pharmaceutical composition” is meant to encompass a composition suitable for administration to a subject, such as a mammal, especially a human. In general, a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a subject compound and a pharmaceutically acceptable excipient. In some embodiments, a pharmaceutically acceptable excipient is other than DMSO.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-c arboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a compound of the invention means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a compound of the invention means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

“Pro-drugs” means any compound that releases an active parent drug according to one or more of the generic formulas shown below in vivo when such prodrug is administered to a mammalian subject. Prodrugs of a compound of one or more of the generic formulas shown below are prepared by modifying functional groups present in the compound of the generic formula in such a way that the modifications may be cleaved in vivo to release the parent compound. Prodrugs include compounds of one or more of the generic formulas shown below wherein a hydroxy, amino, or sulfhydryl group in one or more of the generic formulas shown below is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of one or more of the generic formulas shown below, and the like.

The term “organic group” and “organic radical” as used herein means any carbon-containing group, including hydrocarbon groups that are classified as an aliphatic group, cyclic group, aromatic group, functionalized derivatives thereof and/or various combinations thereof. The term “aliphatic group” means a saturated or unsaturated linear or branched hydrocarbon group and encompasses alkyl, alkenyl, and alkynyl groups, for example.

The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like.

The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group.

The term “alkenyl group” means an unsaturated, linear or branched hydrocarbon group with one or more carbon-carbon double bonds, such as a vinyl group.

The term “alkynyl group” means an unsaturated, linear or branched hydrocarbon group with one or more carbon-carbon triple bonds.

The term “cyclic group” means a closed ring hydrocarbon group that is classified as an alicyclic group, aromatic group, or heterocyclic group.

The term “alicyclic group” means a cyclic hydrocarbon group having properties resembling those of aliphatic groups.

The term “aromatic group” or “aryl group” means a mono- or polycyclic aromatic hydrocarbon group, and may include one or more heteroatoms, and which are further defined below.

The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl sub stituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Halogens of particular interest include chloro and bromo groups.

The term “haloalkyl” refers to an alkyl group as defined above that is substituted by one or more halogen atoms. The halogen atoms may be the same or different.

The term “dihaloalkyl ” refers to an alkyl group as described above that is substituted by two halo groups, which may be the same or different.

The term “trihaloalkyl” refers to an alkyl group as describe above that is substituted by three halo groups, which may be the same or different.

The term “perhaloalkyl” refers to a haloalkyl group as defined above wherein each hydrogen atom in the alkyl group has been replaced by a halogen atom.

The term “perfluoroalkyl” refers to a haloalkyl group as defined above wherein each hydrogen atom in the alkyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like. The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The terms “substituted phenyl” or “substituted aryl” specifies a phenyl group or an aryl group substituted with one or more moieties, and in some instances one, two, or three moieties, chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C₁ to C₆ alkyl)carboxamide, protected N-(C₁ to C₆ alkyl)carboxamide, N,N-di(C₁ to C₆ alkyl)carboxamide, trifluoromethyl, N-((C₁ to C₆ alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also, as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to C₄ alkoxy group, similarly, “lower alkylthio” means a C₁ to C₄ alkylthio group.

The term “(monosubstituted)amino” refers to an amino group with one substituent chosen from the group consisting of phenyl, substituted phenyl, alkyl, substituted alkyl, C₁ to C₄ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different.

The term “heteroaryl(alkyl)” denotes an alkyl group as defined above, substituted at any position by a heteroaryl group, as above defined.

“Optional” or “optionally” means that the subsequently described event, circumstance, feature, or element may, but need not, occur, and that the description includes instances where the event or circumstance occurs and instances in which it does not. For example, “heterocyclo group optionally mono- or di-substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

A subject compound may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

“In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a mitochondrial aldehyde dehydrogenase-2 agonist” includes a plurality of such agonists and reference to “the pharmaceutical composition” includes reference to one or more pharmaceutical compositions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of protecting and expanding hematopoietic cells. The present disclosure also provides methods of increasing the potency of hematopoietic cells. In some cases, the hematopoietic cells are hematopoietic stem cells (HSC). In some cases, the hematopoietic cells are hematopoietic stem and progenitor cells (HSPCs). The present disclosure provides methods of increasing the number of hematopoietic cells in an individual. The present disclosure also provides methods of increasing the potency of the hematopoietic cells in an individual. The methods generally involve contacting hematopoietic cells in vivo, in vitro, or ex vivo with an aldehyde dehydrogenase-2 (ALDH2) agonist. Contacting the hematopoietic cells with the ALDH2 agonist protects and increases the number of hematopoietic cells and generates an expanded population of hematopoietic cells. Contacting the hematopoietic cells with the ALDH2 agonist also increases the potency of hematopoietic cells. These methods find use in a variety of medical applications in which protection and expansion of hematopoietic cells is desired. These methods also find use in a variety of medical applications where increased potency of hematopoietic cells is desired.

Functional properties of HSCs include the potential for both pluripotentiality (the ability to give rise to all blood cell types) and self-renewal (the ability of at least one daughter cell or both to continue to be stem cells). Engraftment after HSC transplant (HSCT) experimentally is one way to test both of these properties. A specialized version of the HSCT model is to experimentally transplant a recipient with HSCs from two different donors, which can be genetically distinguished by a marker gene or protein. The inventors have shown herein that in such competitive repopulation experiments, it is possible to determine whether the HSCs from one donor are more potent than those from another donor, by measuring whether more or less of the resultant blood cells developing from the transplanted HSC are derived from one donor vs the other.

### Methods of Protecting, Expanding and Increasing the Potency of Hematopoietic Cells

The present disclosure provides methods of protecting and expanding hematopoietic cells, the methods including contacting a starting population of hematopoietic cells with a therapeutically effective amount of at least one ALDH2 agonist. The present disclosure also provides methods of increasing the potency of hematopoietic cells, the methods including contacting a starting population of hematopoietic cells with a therapeutically effective amount of at least one ALDH2 agonist, wherein the contacting increases the potency of the HSCs relative to untreated HSCs. Suitable ALDH2 agonists include compounds that increase the activity of ALDH2.

Accordingly, in one embodiment there is provided a method of treating hematopoietic cells, the method comprising contacting a starting population of hematopoietic cells with a therapeutically effective amount of at least one ALDH2 agonist, wherein the contacting results in one or more of protecting, expanding and increasing the potency of the contacted hematopoietic cells relative to the starting population of hematopoietic cells.

In one embodiment the hematopoietic cells comprise hematopoietic stem cells (HSC). In one embodiment the hematopoietic cells comprise hematopoietic stem and progenitor cells (HSPC s). According to one embodiment, a subject method involves contacting hematopoietic cells in vitro, in vivo, or ex vivo with an effective amount of an ALDH2 agonist (e.g., a natural or synthetic ALDH2 agonist), wherein the contacting increases the number of hematopoietic cells by at least about 10%, thereby generating an expanded population of hematopoietic cells. In certain aspects, the ALDH2 agonist increases the number of hematopoietic cells by at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the number of hematopoietic cells not contacted with the

ALDH2 agonist. In some cases, the ALDH2 agonist expands the number of hematopoietic cells from 2-fold to 4-fold. In some cases, the ALDH2 agonist expands the number of hematopoietic cells 2-fold or more. In some cases, the ALDH2 agonist expands the number of hematopoietic cells 4-fold or more. According to another embodiment, a subject method involves contacting hematopoietic cells in vitro, in vivo, or ex vivo with an effective amount of an ALDH2 agonist (e.g., a natural or synthetic ALDH2 agonist), wherein the contacting protects the hematopoietic cells from the adverse effects of radiation, chemotherapy damaging toxins and the like. In some cases, contacting the hematopoietic cells with an effective amount of an ALDH2 agonist protects the number of hematopoietic cells so that at least about 10% more of the hematopoietic cells are protected compared to a population of hematopoietic cells not contacted with an ALDH2 agonist. In certain aspects, the ALDH2 agonist protects the number of hematopoietic cells so that at least 10% more, at least 15% more, at least 20% more, at least 25% more, at least 40% more, at least 50% more, at least 75% more, at least 80% more, at least about 85% more, at least 90% more, at least 95% more, at least 98% more, or over 98% more, of the hematopoietic cells are protected compared to a population of hematopoietic cells not contacted with an ALDH2 agonist. In some cases, the number of hematopoietic cells protected is at least 2-fold, at least 2.5-fold, at least 3-fold, at least 3.5-fold, at least 4-fold or greater than 4-fold, more than in a population of hematopoietic cells not contacted with an ALDH2 agonist.

According to another embodiment, a subject method involves contacting hematopoietic cells in vitro, in vivo, or ex vivo with an effective amount of an ALDH2 agonist (e.g., a natural or synthetic ALDH2 agonist), wherein the contacting increases the potency of the hematopoietic cells compared to untreated hematopoietic cells. In some cases, contacting the hematopoietic cells with an effective amount of an ALDH2 agonist increases the potency of hematopoietic cells by at least about 10% compared to a population of hematopoietic cells not contacted with an ALDH2 agonist. In certain aspects, the ALDH2 agonist increases the potency of hematopoietic cells by at least 10% more, at least 15% more, at least 20% more, at least 25% more, at least 40% more, at least 50% more, at least 75% more, at least 80% more, at least about 85% more, at least 90% more, at least 95% more, at least 98% more, or over 98% more, compared to a population of hematopoietic cells not contacted with an ALDH2 agonist. In some cases, the potency of the hematopoietic cells is at least 2-fold, at least 2.5-fold, at least 3-fold, at least 3.5-fold, at least 4-fold or greater than 4-fold, greater than hematopoietic cells not contacted with an ALDH2 agonist.

A subject method can be carried out in vivo. For example, a therapeutically effective amount of at least one ALDH2 agonist is administered to an individual in need thereof. The subject methods are useful in the medical treatment of any disease that involves hematopoietic stem cells. Examples include but are not limited to, cancer, bone marrow failure conditions, congenital diseases (e.g. sickle cell anemia and thalassemia), lupus, acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin's lymphoma, Hodgkin's disease, aplastic anemia, pure red cell aplasia, hemoglobinuria, Fanconi anemia, thalassemia, sickle cell anemia, Wiskott-Aldrich syndrome, inborn errors of metabolism (e.g. Gaucher disease).

In one embodiment, increasing the potency, protecting and expanding the number of hematopoietic cells is useful for treating an individual who has undergone, or is about to undergo, radiation treatment for a cancer. Expanding the number of hematopoietic cells in an individual provides for an increase in the number of hematopoietic cells in the individual following radiation treatment. For example, a subject method provides for an increase in the number of hematopoietic cells in an individual following radiation treatment for a cancer of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the number of hematopoietic cells in the individual in the absence of treatment with a subject method. Further, a subject method provides for protection of the hematopoietic cells in an individual following radiation treatment for a cancer, such that at least 10%, at least 15%, at least 20%, at least 25%, at least 40%, at least 50%, at least 75%, at least 2-fold, at least 2.5-fold, at least 3-fold, at least 3.5-fold, at least 4-fold or more of the hematopoietic cells are protected when compared to the hematopoietic cells in the individual in the absence of treatment with a subject method. Still further, a subject method provides for an increase in the potency of hematopoietic cells in an individual following radiation treatment for a cancer of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the potency of hematopoietic cells in the individual in the absence of treatment with a subject method.

In another embodiment, increasing the potency, protecting and expanding the number of hematopoietic cells is useful for treating an individual who has undergone, or is about to undergo chemotherapy for a cancer. Expanding the number of hematopoietic cells in an individual provides for an increase in the number of hematopoietic cells in the individual following chemotherapy treatment. For example, a subject method provides for an increase in the number of hematopoietic cells in an individual following chemotherapy treatment for a cancer of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the number of hematopoietic cells in the individual in the absence of treatment with a subject method. Further, a subject method provides for protection of the hematopoietic cells in an individual following chemotherapy treatment for a cancer, such that at least 10%, at least 15%, at least 20%, at least 25%, at least 40%, at least 50%, at least 75%, at least 2-fold, at least 2.5-fold, at least 3-fold, at least 3.5-fold, at least 4-fold or more of the hematopoietic cells are protected when compared to the number of hematopoietic cells in the individual in the absence of treatment with a subject method. Still further, a subject method provides for an increase in potency of hematopoietic cells in an individual following chemotherapy treatment for a cancer of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the potency of hematopoietic cells in the individual in the absence of treatment with a subject method.

In another embodiment, increasing the potency, protecting and expanding the number of hematopoietic cells is useful for treating an individual who has been exposed to one or more damaging toxin. Expanding the number of hematopoietic cells in an individual provides for an increase in the number of hematopoietic cells in the individual following exposure to one or more damaging toxins. For example, a subject method provides for an increase in the number of hematopoietic cells in an individual following exposure to one or more damaging toxins of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the number of hematopoietic cells in the individual in the absence of treatment with a subject method. Further, a subject method provides for protection of hematopoietic cells in an individual following exposure to one or more damaging toxins, such that at least 10%, at least 15%, at least 20%, at least 25%, at least 40%, at least 50%, at least 75%, at least 2-fold, at least 2.5-fold, at least 3-fold, at least 3.5-fold, at least 4-fold or more of the hematopoietic cells are protected when compared to the number of hematopoietic cells in the individual in the absence of treatment with a subject method. Still further, a subject method provides for an increase in potency of hematopoietic cells in an individual following exposure to one or more damaging toxins of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the potency of hematopoietic cells in the individual in the absence of treatment with a subject method.

In some cases, increasing the potency, protecting and expanding the number of hematopoietic cells is useful for treating an individual who has a genetic disease that leads to HSC damage, bone marrow failure, an autoimmune disease, or development of hematologic malignancies, including, but not limited to, lymphoid and myeloid leukemia, Fanconi anemia, aplastic anemia, Hunter syndrome, Wiskott-Aldrich syndrome, β-thalassemia, or neuroblastoma. For example, a subject method provides for an increase in the number of hematopoietic cells by at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the number of hematopoietic cells in the individual in the absence of treatment with a subject method. Further, a subject method provides for protection of the hematopoietic cells, such that at least 10%, at least 15%, at least 20%, at least 25%, at least 40%, at least 50%, at least 75%, at least 2-fold, at least 2.5-fold, at least 3-fold, at least 3.5-fold, at least 4-fold or more of the hematopoietic cells are protected when compared to the number of hematopoietic cells in the individual in the absence of treatment with a subject method. Still further, a subject method provides for an increase in the potency of hematopoietic cells by at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the potency of hematopoietic cells in the individual in the absence of treatment with a subject method.

In some cases, increasing the potency, protecting and expanding the number of hematopoietic cells is useful for treating an individual who is a stem cell transplant donor. Expanding the number of hematopoietic cells in an individual provides for an increase in the number of hematopoietic cells in the individual preceding stem cell donation. For example, a subject method provides for an increase in the number of hematopoietic cells in an individual preceding stem cell donation of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the number of hematopoietic cells in the individual in the absence of treatment with a subject method. Further, a subject method provides for protection of the hematopoietic cells in an individual preceding stem cell donation, such that at least 10%, at least 15%, at least 20%, at least 25%, at least 40%, at least 50%, at least 75%, at least 2-fold, at least 2.5-fold, at least 3-fold, at least 3.5-fold, at least 4-fold or more of the hematopoietic cells are protected when compared to the number of hematopoietic cells in the individual in the absence of treatment with a subject method. Still further, a subject method provides for an increase in the potency of hematopoietic cells in an individual preceding stem cell donation of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the potency of hematopoietic cells in the individual in the absence of treatment with a subject method.

In some embodiments, the treated hematopoietic cells are transplanted into an individual with a hematological or genetic disease, such as lymphoid and myeloid leukemia, lymphoma, Fanconi anemia, aplastic anemia, severe combined immune deficiency, chronic granulomatous disease, congenital neutropenia, Hunter syndrome, Wiskott-Aldrich syndrome, β-thalassemia, sickle cell disease, or neuroblastoma.

As noted above, in some cases, a subject method is carried out in vitro. Thus, a starting population of hematopoietic cells can be contacted with an ALDH2 agonist in vitro to increase the potency of, protect and expand the number of hematopoietic cells. In some embodiments, contacting the hematopoietic cells with an ALDH2 agonist in vitro generates and protects an expanded population of hematopoietic cells that are being genetically modified by a virus, a plasmid or CRISPR mediated gene therapy, or genomic editing. For example, a subject in vitro method provides for an increase in the number of hematopoietic cells of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the number of hematopoietic cells in the absence of contacting with an ALDH2 agonist. In some embodiments, contacting the hematopoietic cells with an ALDH2 agonist in vitro increases the potency of the hematopoietic cells that are being genetically modified by a virus, a plasmid or CRISPR mediated gene therapy, or genomic editing. For example, a subject in vitro method provides for an increase in the potency of hematopoietic cells of at least 10%, at least 15%, at least 20%, at least 25%, at least 40%, at least 50%, at least 75%, at least 2-fold, at least 2.5-fold, at least 3-fold, at least 3.5-fold, at least 4-fold or more than 4-fold, compared to the potency of hematopoietic cells in the absence of contacting with an ALDH2 agonist.

In some embodiments, the starting population of hematopoietic cells can be contacted in vitro with an antibody that specifically recognizes a marker associated with hematopoietic cells. In some cases, the marker is selected from CD34, CD90, c-Kit, CD133, CD38, and combinations thereof.

In some cases, a subject method is carried out ex vivo, e.g., a starting population of hematopoietic cells are obtained from a donor individual, the hematopoietic cells are treated ex vivo by contacting the hematopoietic cells with one or more ALDH2 agonists, to produce an ex vivo contacted population of hematopoietic cells. In some cases, the ex vivo contacting results in one or more of protecting, expanding and increasing the potency of the contacted hematopoietic cells relative to the starting population of hematopoietic cells. In some cases, the ex vivo population of hematopoietic cells have increased potency compared to the starting population of hematopoietic cells. In some cases, the ex vivo population of hematopoietic cells are expanded compared to the starting population of hematopoietic cells. In some cases, the ex vivo population of hematopoietic cells are protected compared to the starting population of hematopoietic cells. In some cases, the ex vivo population of hematopoietic cells are expanded, protected and have increased potency compared to the starting population of hematopoietic cells.

In one embodiment the hematopoietic cells include hematopoietic stem cells (HSC). In another embodiment the hematopoietic cells include hematopoietic stem and progenitor cells (HSPCs). In some cases, the hematopoietic cells are isolated from umbilical cord blood. In some cases, the hematopoietic cells are isolated from bone marrow. In some cases, the hematopoietic cells are isolated from peripheral blood.

In one embodiment, the ex vivo expanded population of hematopoietic cells is introduced into a recipient individual, e.g., an individual who is undergoing chemotherapy treatment and/or radiation treatment for the cancer. In some instances, the donor individual is the same as the recipient individual, e.g., hematopoietic cells are obtained from the donor individual before the donor individual undergoes chemotherapy and/or radiation treatment for a cancer, the hematopoietic cells are expanded ex vivo, as described above, and the ex vivo expanded donor hematopoietic cell population is introduced into the donor individual (who is now the recipient) after the donor has undergone chemotherapy treatment and/or radiation treatment for the cancer (i.e. autologous transplant). In other embodiments, the donor individual and the recipient individual are not the same individual (i.e. allogenic transplant). In some embodiments, the expanded hematopoietic cells are transplanted into an individual with a hematological or genetic disease, such as lymphoid and myeloid leukemia, Fanconi anemia, aplastic anemia, Hunter syndrome, Wiskott-Aldrich syndrome, β-thalassemia, or neuroblastoma.

In another embodiment, the ex vivo protected population of hematopoietic cells is introduced into a recipient individual, e.g., an individual who is undergoing chemotherapy treatment and/or radiation treatment for the cancer. In some instances, the donor individual is the same as the recipient individual, e.g., hematopoietic cells are obtained from the donor individual before the donor individual undergoes chemotherapy and/or radiation treatment for a cancer, the hematopoietic cells are protected ex vivo, as described above, and the ex vivo protected donor hematopoietic cell population is introduced into the donor individual (who is now the recipient) after the donor has undergone chemotherapy treatment and/or radiation treatment for the cancer (i.e. autologous transplant). In other embodiments, the donor individual and the recipient individual are not the same individual (i.e. allogenic transplant). In some embodiments, the protected hematopoietic cells are transplanted into an individual with a hematological or genetic disease, such as lymphoid and myeloid leukemia, Fanconi anemia, aplastic anemia, Hunter syndrome, Wiskott-Aldrich syndrome, β-thalassemia, or neuroblastoma.

In yet another embodiment, the ex vivo population of hematopoietic cells having increased potency is introduced into a recipient individual, e.g., an individual who is undergoing chemotherapy treatment and/or radiation treatment for the cancer. In some instances, the donor individual is the same as the recipient individual, e.g., hematopoietic cells are obtained from the donor individual before the donor individual undergoes chemotherapy and/or radiation treatment for a cancer, the hematopoietic cells are treated ex vivo to increase the potency of the hematopoietic cells, as described above, and the ex vivo donor hematopoietic cell population with increased potency is introduced into the donor individual (who is now the recipient) after the donor has undergone chemotherapy treatment and/or radiation treatment for the cancer (i.e. autologous transplant). In other embodiments, the donor individual and the recipient individual are not the same individual (i.e. allogenic transplant). In some embodiments, the hematopoietic cells with increased potency are transplanted into an individual with a hematological or genetic disease, such as lymphoid and myeloid leukemia, Fanconi anemia, aplastic anemia, Hunter syndrome, Wiskott-Aldrich syndrome, β-thalassemia, or neuroblastoma.

### Isolation and Maintenance of Hematopoietic Cells

A number of approaches for isolating and culturing hematopoietic cells are known in the art, and any such method can be used to obtain hematopoietic cells for use in a subject method. For example, hematopoietic cells may be isolated and cultured as described by, Ng Y Y, Baert MR, de Haas E F, Pike-Overzet K, Staal F J. Isolation of human and mouse hematopoietic stem cells. Methods Mol Biol. 2009; 506:13-21, or R. I. Dimitrieva, S. V. Anisimov. Optimal protocols of hematopoietic stem cell expansion in vitro. Cell and Tissue Biology, May 2013, Volumn 7, Issue 3, pp 207-211, the disclosures of which are incorporated herein by reference in their entireties.

According to one aspect of the present disclosure, hematopoietic cells isolated from a donor individual is minced and dissociated in an appropriate cell dissociation medium, centrifuged, filtered, and resuspended in a medium with one or more growth factors (e.g. stem cell factor (SCF), flt3 ligand (FL), interleukin-3 (IL3) and interleukin-6 (IL6), and the like), antibiotics, and so forth to support maintenance and viability of the dissociated cells. Optionally, the hematopoietic cells are isolated or enriched from the primary cell suspension. This may be achieved by contacting the donor hematopoietic cells in vitro with a reagent (e.g., an antibody) that specifically recognizes a marker associated with hematopoietic cells, where contacting the hematopoietic cells with the reagent is performed prior to contacting the hematopoietic cells with the ALDH2 agonist.

Useful markers for hematopoietic cells include CD34, CD90, c-Kit, CD49f, ALDH1, and combinations thereof. For example, human and mouse hematopoietic cells may be isolated by selecting for CD34-positive and cKit-positive cells, respectively, e.g., using the EASYSEP™ positive selection kit (STEMCELL Technologies, Inc., Vancouver, BC). Detection of markers such as CD49f can be achieved using antibody specific for the marker, where the antibody can comprise a detectable label. Standard methods such as fluorescence activated cell sorting

(FACS) can be used to isolate the cells. ALDH expression can be detected using ALDEFLUOR® aldehyde dehydrogenase fluorescent detection label. For example, ALDH converts the ALDH substrate, BAAA (BODIPY-aminoacetaldehyde), into the fluorescent product BAA (BODIPY-aminoacetate). Cells expressing high levels of ALDH become brightly fluorescent and can be identified using standard flow cytometry methods and/or isolated by cell sorting. See, e.g., Deng et al. (2010) PLoS One 5:e10277.

In certain aspects, hematopoietic cells (isolated or otherwise) may be maintained in a culture medium prior to being contacted with the ALDH2 agonist. For example, the cells may be maintained in a medium that includes one or more factors that prevents the hematopoietic cells from differentiating into more specialized cells.

According to one embodiment, the donor hematopoietic cells are obtained from an individual (e.g., having a cancer) prior to that individual undergoing chemotherapy treatment or radiation treatment, e.g., radiotherapy to treat a cancer. In other aspects, the donor hematopoietic cells are obtained from an individual other than a recipient individual, e.g., an individual who neither has cancer nor is undergoing chemotherapy treatment or radiation treatment.

Contacting Hematopoietic Cells with an ALDH2 Agonist In Vitro

As noted above, in some cases, a subject method is carried out in vitro. Methods of the present disclosure include contacting hematopoietic cells in vitro with an ALDH2 agonist. In the case of contacting hematopoietic cells with an ALDH2 agonist in vitro, the cell culture medium may be supplemented with an effective amount of the agonist. The cell culture medium may be chosen such that the medium is compatible with the agonist, e.g., the agonist is stable and active in the medium. The medium may be supplemented with one or more components that enhance the stability and/or activity of the ALDH2 agonist.

Contacting Hematopoietic Cells with an ALDH2 Agonist Ex Vivo

In some cases, a subject method is carried out ex vivo, e.g., hematopoietic cells are obtained from a donor individual, the hematopoietic stem cells are treated ex vivo by contacting the hematopoietic cells with one or more ALDH2 agonists, to produce an ex vivo population of donor hematopoietic cells as described herein (e.g., where one or more of expansion, protection and increased potency is generated by contacting the cells with an ALDH2 agonist). The ex vivo treated population of donor hematopoietic cells is introduced into a recipient individual, e.g., an individual who is undergoing chemotherapy treatment or radiation treatment for cancer. An ex vivo treated hematopoietic cell population can be obtained by culturing hematopoietic cells ex vivo in a culture medium comprising one or more ALDH2 agonists, where the culturing can take place for about 4 hours to about 72 hours, e.g., from about 4 hours to about 8 hours, from about 8 hours to about 16 hours, from about 16 hours to about 24 hours, from about 24 hours to about 36 hours, from about 36 hours to about 48 hours, or from about 48 hours to about 72 hours, or more than 72 hours.

In some instances, the donor individual is the same as the recipient individual, in which case the cells are considered autologous. For example, hematopoietic cells are obtained from the donor individual, the hematopoietic cells are treated ex vivo, as described above, and the ex vivo treated donor hematopoietic cell population is introduced into the donor individual (who is now the recipient), e.g. after the donor has undergone radiation treatment or chemotherapy treatment for a cancer.

In other embodiments, the donor individual and the recipient individual are not the same individual, in which case the cells are allogeneic. The donor and the recipient can be human leukocyte antigen (HLA) typed before transplantation, and the closest HLA match identified as a suitable donor.

In some embodiments, the treated hematopoietic cells are transplanted into an individual with a hematological or genetic disease, such as lymphoid and myeloid leukemia, lymphoma, Fanconi anemia, aplastic anemia, Hunter syndrome, severe combined immune deficiency, Wiskott-Aldrich syndrome, chronic granulomatous disease, congenital neutropenia, β-thalassemia, sickle cell disease, or neuroblastoma.

Introducing Hematopoietic Cells into a Recipient Individual

As noted above, methods of the present disclosure optionally include introducing a treated population of hematopoietic cells (e.g., where the treatment results in one or more of expansion, protection and increased potency, generated by contacting the cells with an ALDH2 agonist) into a recipient individual (e.g., a human). Introduction of the treated hematopoietic cells is useful in a variety of applications, including treatment of individuals undergoing chemotherapy treatment for cancer, or radiation treatment for cancer, or individuals who have been exposed to damaging toxins. Introduction of the treated hematopoietic cells to a cancer patient make it possible for the patient to receive higher doses of chemotherapy and/or radiation therapy than a patient without treatment with a treated population of hematopoietic cells. Introduction of the treated hematopoietic cells is also useful in the treatment of individuals who have a genetic disease that leads to HSC damage, bone marrow failure, an autoimmune disease, or development of hematologic malignancies. In certain cases, the treated hematopoietic cells are transplanted into an individual with a hematological or genetic disease, such as lymphoid and myeloid leukemia, lymphoma, Fanconi anemia, aplastic anemia, Hunter syndrome, severe combined immune deficiency, Wiskott-Aldrich syndrome, chronic granulomatous disease, congenital neutropenia, β-thalassemia, sickle cell disease, or neuroblastoma.

In one embodiment, the cells to be introduced into the recipient individual are provided as a suspension, which may be a single cell suspension, or a suspension of small clumps of cells, and which are distinguished from solid tissue grafts, which are implanted and are not injected or infused. The cell suspension is a form that can be injected or infused into a recipient. In another embodiment, the cells are provided as an ex vivo engineered tissue construct. Survival of the cells or tissue may be measured after short periods of time, e.g. after at least about three to about seven days.

The number of hematopoietic cells transplanted into a recipient individual can vary from about 10 to about 10⁸, e.g., from 10 to 10², from about 10² to about 10³, from about 10³ to about 10⁴, from about 10⁴ to about 10⁵, from about 10⁵ to about 10⁶, from about 10⁶ to about 10⁷, or from about 10⁷ to about 10⁸. A population of hematopoietic cells to be introduced into a recipient individual is generally at least about 75%, at least about 80%, at least about 85%, at least about 90%, at least about 95%, at least about 98%, or more than 98%, hematopoietic cells.

The hematopoietic cells to be introduced into the recipient individual may be referred to as a cell transplant, e.g., HSC transplant (HSCT). A cell transplant, as used herein, is the transplantation of one or more donor hematopoietic cells into a recipient body, usually for the purpose of augmenting function of an organ or tissue in the recipient. The donor hematopoietic cells may originate from the recipient, in which case the donor and the recipient are the same individual. In other aspects, the recipient is an individual to whom tissue or cells from another individual (donor), commonly of the same species, has been transferred. When the donor and recipient are not the same individual, the HLA antigens (or MHC antigens), which may be Class I or Class II, generally will be matched, although one or more of the HLA antigens may be different in the donor as compared to the recipient. The graft recipient and donor are generally mammals, e.g., humans. Laboratory animals, such as rodents, e.g. mice, rats, etc. are of interest. The cells may be allogeneic, autologous, or xenogeneic with respect to the recipient.

The cells may be provided as a suspension, which suspension includes one or more survival factors. As used herein, the term “survival factors” refers to biologically active agents that are provided in a formulation for the suspension of cells prior to transplantation. The presence of survival factor(s) enhances the survival of cells after the cells are transferred into the body of a recipient. Survival factors may be utilized as one or a cocktail of factors. In some embodiments, the survival factors are also utilized as culture additives for a period of time prior to transplantation.

The donor hematopoietic cells may be administered in any physiologically acceptable excipient including an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The cells may be introduced by injection, catheter, or the like. The cells may be frozen at liquid nitrogen temperatures and stored for long periods of time, being capable of use on thawing. If frozen, the cells may be stored, e.g., in a 10% dimethylsulfoxide (DMSO), 50% fetal calf serum (FCS) (or other suitable serum or serum substitute), 40% RPMI 1640 medium (or other suitable culture medium).

The cell formulations may be used for tissue reconstitution or regeneration in a human patient or other subject in need of such treatment, e.g., a recipient individual having a cancer who has undergone radiation treatment for the cancer. The cells are administered in a manner that permits them to graft or migrate to the intended tissue site and reconstitute or regenerate the functionally deficient area (e.g., an irradiated area).

The hematopoietic cells may also be genetically modified to enhance survival, control proliferation, and the like. Cells may be genetically altering by transfection or transduction with a suitable vector, homologous recombination, or other appropriate technique, so that they express a gene of interest. For example, cells can be transfected with genes encoding a telomerase catalytic component (TERT), e.g., under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over an 8-16 h period, and then exchanged into growth medium for 1-2 days. Genetically altered cells are selected using a drug selection agent such as puromycin, G418, or blasticidin, and then recultured.

### ALDH2 Agonists

A subject method involves use of compounds that function as activators of ALDH2 enzymatic activity. Activators of ALDH2 activity are also referred to herein as ALDH2 agonists.

Mitochondrial aldehyde dehydrogenase-2 (ALDH2) is encoded in the nuclear genome and is transported into mitochondria. ALDH2 is a tetrameric protein composed of four identical subunits, each consisting of 500 amino acid residues. This tetramer can be regarded as a dimer of dimers. The interface between monomers that form a dimer is different and more extensive than the interface between the two dimers that form the tetramer. Each subunit is composed of three main domains: the catalytic domain, the coenzyme or NADtbinding domain, and the oligomerization domain.

Diseases and conditions associated with ALDH2 include, but are not limited to, ischemic stress, chronic free-radical associated diseases, acute free-radical associated diseases, insensitivity to nitroglycerin (e.g., in angina and heart failure), hypertension, diabetes, osteoporosis, cancer, Fanconi anemia, Alzheimer disease, Parkinson disease, alcoholism, alcohol intolerance, alcohol addiction, an alcohol abuse disorder, alcohol intoxication, alcohol dependence, alcohol poisoning, symptoms of alcohol consumption, and narcotic addition.

As disclosed herein, a suitable ALDH2 agonist selectively modulates (e.g., increases) an enzymatic activity of ALDH2. For example, in some embodiments, a suitable ALDH2 agonist increases an enzymatic activity of ALDH2, but does not substantially increase the same enzymatic activity of an ALDH isozyme other than ALDH2, e.g., the ALDH agonist increases an enzymatic activity of an ALDH isozyme other than ALDH2, if at all, by no more than about 15%, e.g., by less than 15%, less than 10%, less than 5%, or less than 1%.

A subject method involves use of an ALDH2 agonist (also referred to as “ALDH2 activator”). A suitable ALDH2 agonist increases an enzymatic activity of an ALDH2 polypeptide by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100% (or two-fold), at least about 2.5-fold, at least about 5-fold, at least about 10-fold, at least about 15-fold, at least about 20-fold, at least about 25-fold, or at least about 50-fold, or greater than 50-fold, when compared to the enzymatic activity of the ALDH2 polypeptide in the absence of the agonist.

In some embodiments, a suitable ALDH2 agonist has an EC₅₀ (half maximal effective concentration) of from about 1 nM to about 1 mM, e.g., from about 1 nM to about 10 nM, from about 10 nM to about 15 nM, from about 15 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 450 nM, from about 450 nM to about 500 nM, from about 500 nM to about 750 nM, from about 750 nM to about 1μM, from about 1μM to about 10 μM, from about 10 ₁1M to about 25 μM, from about 25 ₁1M to about 50 μM, from about 50 ₁1M to about 75 μM, from about 75 ₁1M to about 100 μM, from about 100 ₁1M to about 250 μM, from about 250₁1M to about 500 μM, or from about 500₁1M to about 1 mM.

In some embodiments, a suitable ALDH2 agonist is a compound of generic Formula I, as shown below:

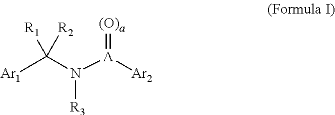

Wherein, R₁, R₂, and R₃ are each independently selected from the group consisting of hydrogen, halogen, aryl, substituted aryl, alkyl, substituted alkyl, alkenyl, substituted alkenyl, alkynyl, substituted alkynyl, cycloalkyl, substituted cycloalkyl, heterocycle, substituted heterocycle, heteroaryl, substituted heteroaryl; A is selected from C and S, wherein a is 1 when A is C and a is 2 when A is S; and Ar₁ and Ar₂ are each independently selected from the group consisting of aryl, substituted aryl, heteroaryl and substituted heteroaryl, or a prodrug, a pharmaceutically acceptable salt, solvate, analog or derivative thereof.

In some embodiments, a suitable ALDH2 agonist is a compound of generic Formula II, as shown below:

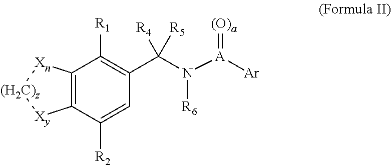

where Xₙ and X_{y} are each independently H, C, N, O, or a halogen; where n is the integer 0 or 1;
where y is the integer 0 or 1;
where (dotted line) is an optional bond; where z is the integer 0, 1, or 2;
where A is C or S, and where a=1 when A=C; and where a=2 when A=S;
where Ar is an unsubstituted or substituted aryl group; and
where R₁ to R₆ is each independently selected from H; a halo (e.g., bromo, fluoro, chloro, iodo); a substituted or unsubstituted phenyl group; an aliphatic group, an alkyl group; a substituted alkyl group; an alkenyl group; an alkynyl group; a substituted or unsubstituted cyclic group; a substituted or unsubstituted heterocyclic group; a substituted or unsubstituted aryl group; and a substituted or unsubstituted heteroaryl group;
or a pro-drug, a pharmaceutically acceptable salt, an analog, or a derivative thereof.

In some embodiments, a suitable ALDH2 agonist is a compound of generic Formula III, as shown below:

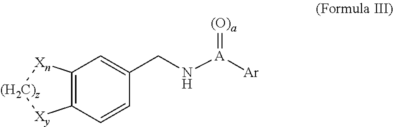

wherein X is O or F;
. . . (dotted line) is an optional bond;
z is the integer 0, 1, or 2, with the provisos that: 1) z=0 when X=F . . . and is not a bond; and 2) when z=0, X=O, . . . is not a bond, and one or more oxygen atoms (X) are present, oxygen is attached to a methyl group;
n is the integer 0 or 1;
y is the integer 0 or 1;
A=C or S, and where a=1 when A=C; and where a=2 when A=S; and
Ar is a phenyl or thiophene ring; wherein the Ar is optionally substituted at the position(s) ortho to the carbonyl or sulfonyl group by one or more substituents independently selected from methyl, halo, trifluoromethyl, or phenyl; wherein Ar is optionally substituted by a halogen meta or para to the carbonyl or sulfonyl group; and wherein, when Ar is a thiophene ring, the carbonyl or sulfonyl group is attached to a thiophene ring at the 2 or 3 position; or a pro-drug, a pharmaceutically acceptable salt, an analog, or a derivative thereof.

In some embodiments, a suitable ALDH2 agonist is the compound Alda-1:

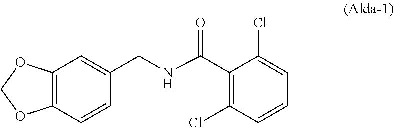 [SMILES: O=C(NCc1ccc2c(c1)OCO2)c1c(Cl)cccc1Cl]

or a pharmaceutically acceptable salt or solvate thereof. In some embodiments, a suitable ALDH2 agonist is a prodrug of Alda-1. In some embodiments, a suitable ALDH2 agonist is a derivative or an analog of Alda-1.
In some embodiments, a subject ALDH2 agonist is Compound 2:

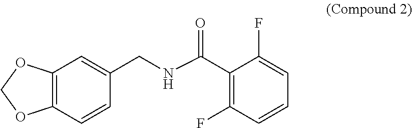 [SMILES: O=C(NCc1ccc2c(c1)OCO2)c1c(F)cccc1F]

or a pharmaceutically acceptable salt or solvate thereof. In some embodiments, a suitable ALDH2 agonist is a prodrug of compound 2. In some embodiments, a suitable ALDH2 agonist is a derivative or an analog of compound 2.

In some embodiments, a subject ALDH2 agonist is Compound 3:

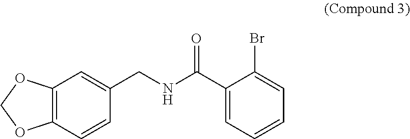 [SMILES: O=C(NCc1ccc2c(c1)OCO2)c1ccccc1Br]

or a pharmaceutically acceptable salt or solvate thereof. In some embodiments, a suitable ALDH2 agonist is a prodrug of compound 3. In some embodiments, a suitable ALDH2 agonist is a derivative or an analog of compound 3.

In some embodiments, a subject ALDH2 agonist is Compound 4:

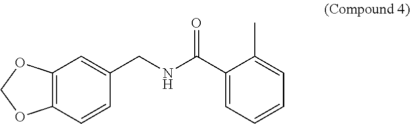 [SMILES: Cc1ccccc1C(=O)NCc1ccc2c(c1)OCO2]

or a pharmaceutically acceptable salt or solvate thereof. In some embodiments, a suitable ALDH2 agonist is a prodrug of compound 3. In some embodiments, a suitable ALDH2 agonist is a derivative or an analog of compound 3.

In some embodiments, a subject ALDH2 agonist has the structure of one of the compounds designated XO-3, XO-4, XO-5, XO-9, XO-28, XO-29, XO-33, XO-36, XO-39, XO-12, XO-13, XO-6, XO-7, XO-8, XO-11, XO-22, XO-25, and XO-26, as shown below:

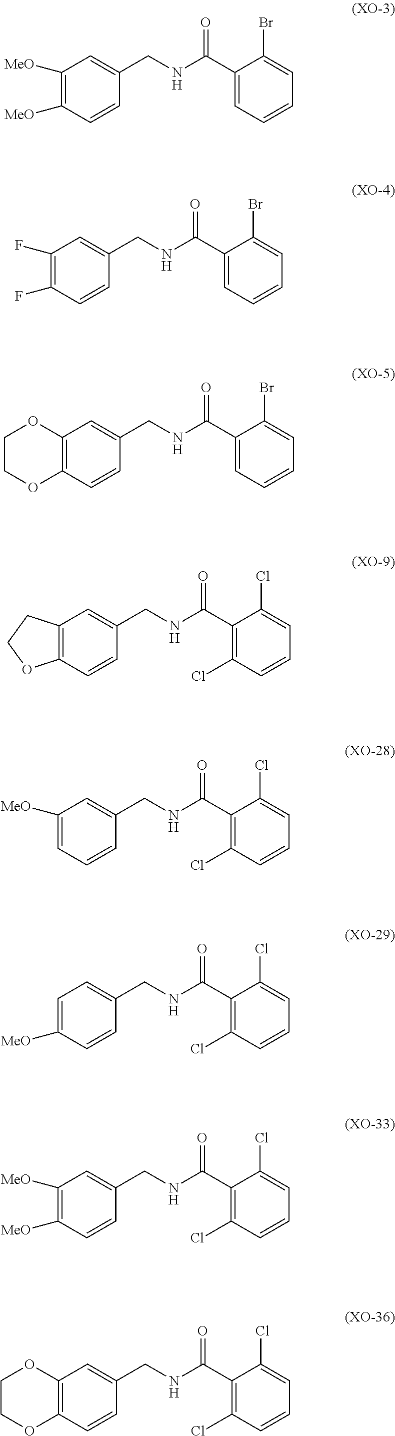

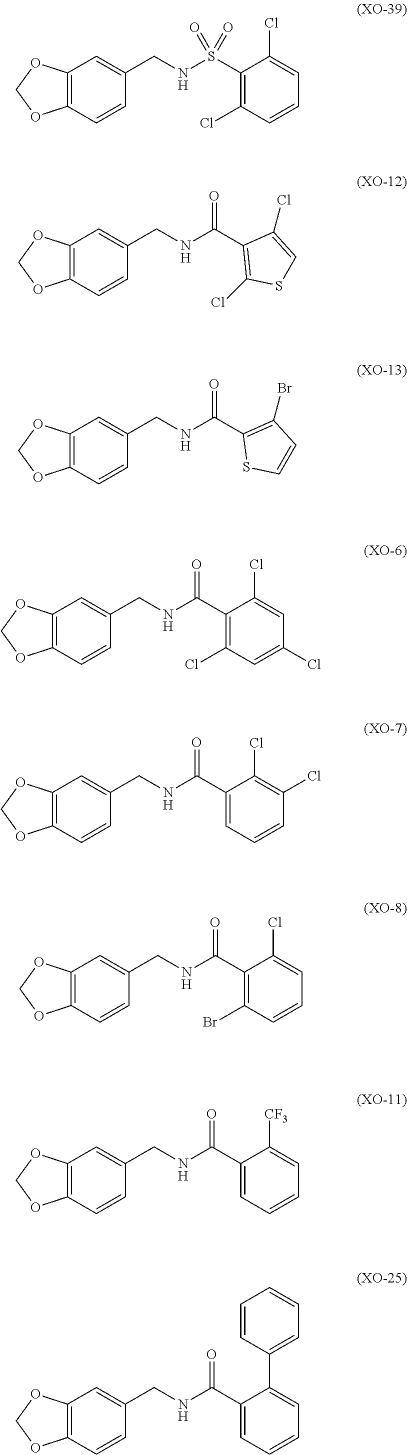

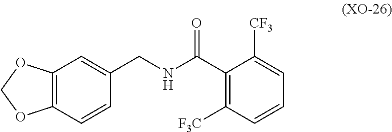 [SMILES: O=C(NCc1ccc2c(c1)OCO2)c1c(C(F)(F)F)cccc1C(F)(F)F]

In some embodiments, a subject ALDH2 agonist is Compound XO-43:

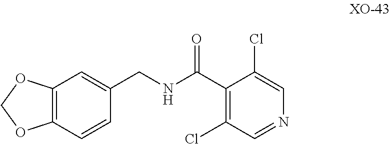 [SMILES: O=C(NCc1ccc2c(c1)OCO2)c1c(Cl)cncc1Cl]

or a pharmaceutically acceptable salt or solvate thereof. In some embodiments, a suitable ALDH2 agonist is a prodrug of compound XO-43. In some embodiments, a suitable ALDH2 agonist is a derivative or an analog of compound XO-43.

In some embodiments, a subject ALDH2 agonist is Compound XO-44:

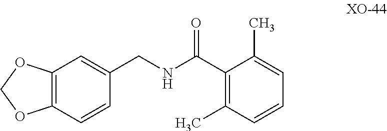 [SMILES: Cc1cccc(C)c1C(=O)NCc1ccc2c(c1)OCO2]

or a pharmaceutically acceptable salt or solvate thereof. In some embodiments, a suitable ALDH2 agonist is a prodrug of compound XO-44. In some embodiments, a suitable ALDH2 agonist is a derivative or an analog of compound XO-44.

In some embodiments, a subject ALDH2 agonist is Compound XO-45:

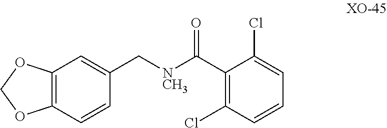 [SMILES: CN(Cc1ccc2c(c1)OCO2)C(=O)c1c(Cl)cccc1Cl]

or a pharmaceutically acceptable salt or solvate thereof. In some embodiments, a suitable ALDH2 agonist is a prodrug of compound XO-45. In some embodiments, a suitable ALDH2 agonist is a derivative or an analog of compound XO-45.

In some embodiments, a subject ALDH2 agonist is Compound XO-46:

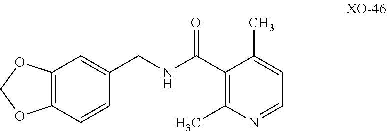 [SMILES: Cc1ccnc(C)c1C(=O)NCc1ccc2c(c1)OCO2]

or a pharmaceutically acceptable salt or solvate thereof. In some embodiments, a suitable ALDH2 agonist is a prodrug of compound XO-46. In some embodiments, a suitable ALDH2 agonist is a derivative or an analog of compound XO-46.

In some embodiments examples of ALDH2 agonists include those disclosed in WO 2015/084731, US applications US2014/323520, US2016/0107996, and U.S. Pat. Nos. 7,560,241, 8,389,522, 8,772,295, 8,906,942, 9,102,651, 9,670,162, 9,545,393 and 8,354,435, the full disclosures of which are herein incorporated by reference.

Whether a compound is an ALDH2 agonist can be readily ascertained. Assays for dehydrogenase activity of ALDH2 are known in the art, and any known assay can be used. Examples of dehydrogenase assays are found in various publications, including, e.g., Sheikh et al. ((1997) J. Biol. Chem. 272:18817-18822); Vallari and Pietruszko (1984) J. Biol. Chem. 259:4922; and Farres et al. ((1994) J. Biol. Chem. 269:13854-13860).

As an example of an assay for dehydrogenase activity, ALDH2 aldehyde dehydrogenase activity is assayed at 25° C. in 50 mM sodium pyrophosphate HC1 buffer, pH 9.0, 100 mM sodium phosphate buffer, pH 7.4, or 50 mM sodium phosphate buffer, pH 7.4, where the buffer includes NAD (e.g., 0.8 mM NAD⁺, or higher, e.g., 1 mM, 2 mM, or 5 mM NAD^{±}) and an aldehyde substrate such as 14 μM propionaldehyde. Reduction of NAD is monitored at 340 nm using a spectrophotometer, or by fluorescence increase using a fluoromicrophotometer. Enzymatic activity can be assayed using a standard spectrophotometric method, e.g., by measuring a reductive reaction of the oxidized form of nicotinamide adenine dinucleotide (NAD⁺) to its reduced form, NADH, at 340 nm, as described in US 2005/0171043; and WO 2005/057213, and as depicted schematically in FIG. 4. In an exemplary assay, the reaction is carried out at 25° C. in 0.1 sodium pyrophosphate (NaPPi) buffer, pH 9.0, 2.4 mM NAD and 10 mM acetaldehyde as the substrate. Enzymatic activity is measured by a reductive reaction of NAD⁺ to NADH at 340 nm, as described in US 2005/0171043; and WO 2005/057213. Alternatively, the production of NADH can be coupled with another enzymatic reaction that consumes NADH and that provides for a detectable signal. An example of such an enzymatic reaction is a diaphorase-based reaction, which reduces resazurin to its oxidized fluorescent compound resorufin, as described in US 2005/0171043; and WO 2005/057213. Detection of fluorescent resorufin at 590 nm provides amplified and more sensitive signals for any change in ALDH2 aldehyde dehydrogenase enzymatic activity.

Whether a compound increases an esterase activity of ALDH2 can be determined using any known assay for esterase activity. For example, esterase activity of ALDH2 can be determined by monitoring the rate of p-nitrophenol formation at 400 nm in 25 mM N,N-Bis(2-hydroxyethyl)-2-amino ethanesulfonic acid (BES) (pH 7.5) with 800 μM p-nitrophenyl acetate as the substrate at room temperature in the absence or presence of added NAD+. A pH-dependent molar extinction coefficient of 16 mM-1 cm-1 at 400 nm for nitrophenol can be used. See, e.g., Larson et al. (2007) J. Biol. Chem. 282:12940). Esterase activity of ALDH2 can be determined by measuring the rate of p-nitrophenol formation at 400 nm in 50 mM Pipes (pH 7.4) with 1 mM p-nitrophenylacetate as the substrate. A molar extinction coefficient of 18.3×103 M-1cm-1 at 400 nm for p-nitrophenolate can be used for calculating its rate of formation. See, e.g., Ho et al. (2005) Biochemistry 44:8022).

Whether a compound increases a reductase activity of ALDH2 can be determined using any known assay for reductase activity. A reductase activity of ALDH2 can be determined by measuring the rate of 1,2-glyceryl dinitrate and 1,3-glyceryl dinitrate formation using a thin layer chromatography (TLC) or liquid scintillation spectrometry method, using a radioactively labeled substrate. For example, 0.1 mM or 1 mM GTN (glyceryl trinitrate) is incubated with the assay mixture (1 ml) containing 100 mM KPi (pH 7.5), 0.5 mM EDTA, 1 mM NADH, 1 mM NADPH in the presence ALDH2. After incubation at 37° C. for about 10 minutes to about 30 minutes, the reaction is stopped and GTN and its metabolites are extracted with 3×4 ml ether and pooled, and the solvent is evaporated by a stream of nitrogen. The final volume is kept to less than 100 ml in ethanol for subsequent TLC separation and scintillation counting. See, e.g., Zhang and Stamler (2002) Proc. Natl. Acad. Sci. USA 99:8306.

In some embodiments, a suitable ALDH2 agonist is pure, e.g., at least 80%, at least about 90% pure, at least about 98% pure, or at least about 99% pure, by weight.

### Natural Extracts

The present disclosure also provides for use of ALDH2 agonists in natural extracts, e.g., extracts of plants and other organisms that naturally contain an ALDH2 agonist. Natural formulations and extracts can comprise an ALDH2 agonist in an amount by weight of from about 0.01% to about 30%, or from about 30% to about 80%, e.g., from about 0.01% to about 0.05%, from about 0.05% to about 0.1%, from about 0.1% to about 0.5%, from about 0.5% to about 1%, from about 1% to about 2.5%, from about 2.5% to about 5%, from about 5% to about 7.5%, from about 7.5% to about 10%, from about 10% to about 12.5%, from about 12.5% to about 15%, from about 15% to about 20%, from about 20% to about 25%, or from about 25% to about 30%. In some embodiments, a suitable natural formulation or natural extract comprises an ALDH2 agonist in an amount by weight of from about 30% to about 35%, from about 35% to about 40%, from about 40% to about 45%, from about 45% to about 50%, from about 50% to about 60%, from about 60% to about 70%, or from about 70% to about 80%. As used herein, a “natural formulation” or a “natural extract” can include components of a plant or other natural source of an ALDH2 agonist, but does not exclude inclusion of components not normally found in a plant source of an ALDH2 agonist, e.g., the “natural formulation” or “natural extract” can include added components not normally found in a plant source or other natural source of an ALDH2 agonist.

A plant or plant part can be extracted either singly or sequentially with one or more of an aqueous solution, an alcohol, a polar organic solvent, and a non-polar organic solvent. In some embodiments, an ALDH2 agonist is water soluble (hydrophilic) and is present in an aqueous phase of a natural extract. For example, in some embodiments, a plant or plant part is extracted with 100% water. In other embodiments, an ALDH2 agonist is hydrophobic and is present in an organic phase of a natural extract. For example, a plant or a plant part can be extracted with an organic solvent such as ethyl acetate or methylene chloride. In some embodiments, the plant or plant part is extracted with alcohol, e.g., methanol or butanol. In some embodiments, the plant or plant part is extracted with methanol:chloroform (1:1 vol:vol). In some embodiments, the plant or plant part is extracted with methanol:water from 95:5 to 1:1. In some embodiments, the plant or plant part is extracted sequentially with an alcohol, then with an alcohol:chloroform mixture. Polar organic solvents include, e.g., tetrahydrofuran, acetonitrile, acetone, and isopropyl alcohol. In some embodiments, the plant or plant part is extracted with a polar organic solvent. Extraction methods are known in the art, and are described in, e.g., U.S. Pat. Nos. 7,282,150 and 7,172,772.

The natural extract can be obtained by extracting a plant or plant part at a temperature of from about 15° C. to about 20° C., from about 20° C. to about 25° C., from about 25° C. to about 30° C., from about 30° C. to about 35° C., from about 35° C. to about 40° C., from about 40° C. to about 45° C., from about 45° C. to about 50° C., from about 50° C. to about 60° C., from about 60° C. to about 70° C., from about 70° C. to about 80° C., from about 80° C. to about 90° C., or from about 90° C. to about 100° C.

A natural extract includes an extract of a whole plant or one or more parts of a plant, where plant parts include leaves, stems, rhizomes, roots, tubers, bulbs, flowers, bark, seeds, fruit, and the like. Thus, sources of an ALDH2 agonist include, e.g., whole plant or one or more parts of a plant, where plant parts include leaves, stems, rhizomes, tubers, bulbs, roots, flowers, bark, seeds, fruit, and the like. Prior to extraction, the plant or plant part can be subjected to one or more processing steps; e.g., prior to extraction, the plant or plant part can be dried, powdered, frozen, steamed, ground, pulverized, or fermented. Pulverizing can be achieved by carrying out one or more of homogenizing, milling, grinding, chopping, blending, cutting, and tearing.

Combinations of two or more extracts are also contemplated, e.g., extracts of two or more different plant parts from the same plant; extracts from two or more plants of the same genus, where the plants are of two or more different species; extracts from two or more plants of two or more different genuses; a combination of an aqueous extract and an alcohol extract; a combination of an aqueous extract and a polar organic solvent extract; a combination of an aqueous extract and a non-polar organic solvent extract; etc.

A suitable natural extract can be formulated in any form convenient for use, e.g., a lozenge, a capsule, a powder, a liquid solution, a gel, etc. Any of a variety of components can be added to a natural extract, including, e.g., fillers, binders, sweeteners, flavors and other ingredients. Nearly any excipients that are known for use in the preparation of oral dosage pharmaceutical products, or natural supplement products, can be used. Examples of such excipients include without limitation, carbomer, carboxymethylcellulose sodium, cellulose, dextrin, dextrose, ethylcellulose, fructose, gelatin, guar gum, hydroxyethyl cellulose, hydroxypropyl cellulose, hydroxypropyl methylcellulose, glucose, maltodextrin, mannitol, methylcellulose, microcrystalline cellulose, polymethacrylates, povidone, sorbitol, starches, sucrose, sugar, sucralose, stevia, and flavor agents.

### Pharmaceutical Compositions, Dosages, Routes of Administration

In some instances, as discussed above, an ALDH2 agonist can be used to increase the number of hematopoietic cells in vivo, e.g., an effective amount of an ALDH2 agonist is administered to an individual in need thereof. The term “ALDH2 agonist” and “ALDH2 activator” are also referred to herein as “active agent.” For administration to an individual, a suitable ALDH2 agonist is formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H.C. Ansel et al., eds., 7^{th} ed., Lippincott, Williams, & Wilkins; and Handbook of Pharmaceutical Excipients (2000) A.H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The term “unit dosage form,” as used herein, refers to physically discrete units suitable as unitary dosages for human and animal subjects, each unit containing a predetermined quantity of an active agent (e.g., an ALDH2 agonist) calculated in an amount sufficient to produce the desired effect in association with a pharmaceutically acceptable diluent, carrier or vehicle. The specifications for an active agent depend on the particular compound employed and the effect to be achieved, and the pharmacodynamics associated with each compound in the host.

In the subject methods, a suitable ALDH2 agonist may be administered to the host using any convenient means capable of resulting in the desired outcome, e.g., reduction of disease, reduction of a symptom of a disease, etc. Thus, a suitable ALDH2 agonist can be incorporated into a variety of formulations for therapeutic administration. More particularly, a suitable ALDH2 agonist can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

In pharmaceutical dosage forms, a suitable ALDH2 agonist (“active agent”) may be administered in the form of its pharmaceutically acceptable salts, or an active agent may be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycol monomethyl ethers, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the active agent. Similarly, unit dosage forms for injection or intravenous administration may comprise an active agent in a composition as a solution in sterile water, normal saline or another pharmaceutically acceptable carrier.

An active agent can be formulated for administration by injection. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

### Dosages and Dosing

Depending on the subject and condition being treated and on the administration route, an active agent may be administered in dosages of, for example, 0.1 μg to 500 mg/kg body weight per day, e.g., from about 0.1 μg/kg body weight per day to about 1 μg/kg body weight per day, from about 1 μg/kg body weight per day to about 25 μg/kg body weight per day, from about 25 μg/kg body weight per day to about 50 μg/kg body weight per day, from about 50 μg/kg body weight per day to about 100 μg/kg body weight per day, from about 100 μg/kg body weight per day to about 500 μg/kg body weight per day, from about 500 μg/kg body weight per day to about 1 mg/kg body weight per day, from about 1 mg/kg body weight per day to about 25 mg/kg body weight per day, from about 25 mg/kg body weight per day to about 50 mg/kg body weight per day, from about 50 mg/kg body weight per day to about 100 mg/kg body weight per day, from about 100 mg/kg body weight per day to about 250 mg/kg body weight per day, or from about 250 mg/kg body weight per day to about 500 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be about ten times the injection dose. Higher doses may be used for localized routes of delivery.

For example, an ALDH2 activator can be administered in an amount of from about 1 mg to about 1000 mg per dose, e.g., from about 1 mg to about 5 mg, from about 5 mg to about 10 mg, from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

An exemplary dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is in some embodiments one which provides up to about 1 μg to about 1,000 μg or about 10,000 μg of an active agent in a blood sample taken from the individual being treated, about 24 hours after administration of the compound to the individual.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the invention. Similarly, unit dosage forms for injection or intravenous administration may comprise the compound(s) in a composition as a solution in sterile water, normal saline or another pharmaceutically acceptable carrier.

In some embodiments, multiple doses of an active agent are administered. The frequency of administration of a compound (“active agent”) can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid). As discussed above, in some embodiments, an active agent is administered continuously.

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, or from about two months to about four months, or more.

### Routes of Administration

A suitable ALDH2 agonist is administered to an individual using any available method and route suitable for drug delivery, including in vivo and ex vivo methods, as well as systemic and localized routes of administration. Administration can be acute (e.g., of short duration, e.g., a single administration, administration for one day to one week), or chronic (e.g., of long duration, e.g., administration for longer than one week, e.g., administration over a period of time of from about 2 weeks to about one month, from about one month to about 3 months, from about 3 months to about 6 months, or more).

Conventional and pharmaceutically acceptable routes of administration include intranasal, intramuscular, intratracheal, subcutaneous, intradermal, transdermal, sublingual, topical application, intravenous, ocular (e.g., topically to the eye, intravitreal, etc.), rectal, nasal, oral, and other enteral and parenteral routes of administration. Routes of administration may be combined, if desired, or adjusted depending upon the agent and/or the desired effect. The compound can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, ocular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of a suitable ALDH2 agonist through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

### Treatment Methods

The present disclosure provides various treatment methods, generally involving administering to an individual in need thereof an effective amount of an ALDH2 agonist and/or an expanded population of hematopoietic cells (e.g., hematopoietic cells expanded in vitro or ex vivo by contacting the hematopoietic cells with an ALDH2 agonist).

Local and/or Systemic ALDH2 Agonist Administration

As noted above, a variety of diseases require treatment with agents which are preferentially cytotoxic to dividing cells. For example, individuals with a cancer commonly undergo chemotherapy or radiotherapy (RT) which treatments are commonly cytotoxic to the hematopoietic cells. Treatment methods of the present disclosure may include in vivo activation of the ALDH2 enzymes and consequently increased potency, protection and expansion of the hematopoietic cells of an individual with a disease, e.g. an individual with a cancer undergoing chemotherapy treatment or radiation treatment for the cancer. The methods may include administering an ALDH2 agonist systemically (e.g., by oral, intravenous, or other systemic administration) or locally (e.g., by local injection and/or topical application at a target site of a composition that includes a modulator of ALDH2 activity). According to one embodiment of the present disclosure, the ALDH2 agonist may be administered (e.g., systemically and/or locally) before the individual with a cancer undergoes radiation therapy or chemotherapy treatment. In another embodiment, the ALDH2 agonist may be administered (e.g., systemically and/or locally) after the individual with a cancer undergoes radiation therapy or chemotherapy treatment. In yet another embodiment, the ALDH agonist is administered before and after the individual undergoes radiation therapy or chemotherapy treatment. In certain embodiments, the ALDH2 agonist is administered continuously for a period of time before the individual is subjected to radiation therapy or chemotherapy treatment. In certain embodiments, the ALDH2 agonist is administered continuously for a period of time after the individual is subjected to radiation therapy or chemotherapy treatment. In some cases, the ALDH2 agonist is administered continuously for a period of time before and after the individual undergoes radiation therapy or chemotherapy.

As noted above, in some embodiments, an ALDH2 agonist is administered as a “pretreatment” to an individual before the individual undergoes radiation treatment or chemotherapy treatment, e.g., from about 1 hour to about 1 week before the radiation treatment or chemotherapy treatment, e.g., from about 1 hour to about 2 hours, from about 2 hours to about 4 hours, from about 4 hours to about 8 hours, from about 8 hours to about 12 hours, from about 12 hours to about 16 hours, from about 16 hours to about 24 hours, from about 24 hours to about 36 hours, from about 36 hours to about 48 hours, from about 48 hours to about 72 hours, or from about 72 hours to about 1 week preceding the radiation treatment.

Pretreatment with an ALDH2 agonist is useful, for example, to expand the number of hematopoietic cells in vivo, such that the probability of a sufficient number of stem cells surviving the radiation or chemotherapy treatment is increased. The above situation is only one example of a circumstance when a subject would benefit from pretreatment with a suitable ALDH2 agonist. In another example, pretreatment with an ALDH2 agonist is useful to increase the potency of hematopoietic cells in vivo, such that the probability of a sufficient number of stem cells surviving the radiation or chemotherapy treatment is increased.

In some embodiments, a suitable ALDH2 agonist is administered after radiation therapy or chemotherapy treatment. For example, a suitable ALDH2 agonist administered after radiation treatment or chemotherapy treatment is effective for mitigating the adverse effects of the radiation treatment on the hematopoietic cells. In some embodiments, a suitable ALDH2 agonist is administered within 1 minute to within 15 hours, e.g., from about 1 minute to about 5 minutes, from about 5 minutes to about 10 minutes, from about 10 minutes to about 15 minutes, from about 15 minutes to about 30 minutes, from about 30 minutes to about 60 minutes, from about 60 minutes to about 2 hours, from about 2 hours to about 4 hours, from about 4 hours to about 8 hours, from about 8 hours to about 12 hours, or from about 12 hours to about 15 hours, following the ischemic event. In some embodiments, an increased concentration of an ALDH2 agonist is maintained in the plasma for at least several hours to several days following the radiation treatment or chemotherapy treatment.

For example, in some embodiments, a suitable ALDH2 agonist is administered to an individual with a cancer within 1 minute to within 15 hours, e.g., from about 1 minute to about 5 minutes, from about 5 minutes to about 10 minutes, from about 10 minutes to about 15 minutes, from about 15 minutes to about 30 minutes, from about 30 minutes to about 60 minutes, from about 60 minutes to about 2 hours, from about 2 hours to about 4 hours, from about 4 hours to about 8 hours, from about 8 hours to about 12 hours, or from about 12 hours to about 15 hours, following radiation treatment or chemotherapy treatment.

The methods may include administering an ALDH2 agonist systemically (e.g., by oral, intravenous, or other systemic administration) or locally (e.g., by local injection and/or topical application at a target site of a composition that includes a modulator of ALDH2 activity) to an individual who has been exposed to one or more damaging toxins.

The methods may include administering an ALDH2 agonist systemically (e.g., by oral, intravenous, or other systemic administration) or locally (e.g., by local injection and/or topical application at a target site of a composition that includes a modulator of ALDH2 activity) to an individual with a genetic disease that leads to HSC damage, bone marrow failure, an autoimmune disease, or development of hematologic malignancies.

The methods may also include administering an ALDH2 agonist systemically (e.g., by oral, intravenous, or other systemic administration) or locally (e.g., by local injection and/or topical application at a target site of a composition that includes a modulator of ALDH2 activity) to an individual who is a stem cell transplant donor. In some embodiments, an ALDH2 agonist is administered to an individual for a period of time preceding stem cell donation, e.g., from about 1 hour to about 1 week before stem cell donation, e.g., from about 1 hour to about 2 hours, from about 2 hours to about 4 hours, from about 4 hours to about 8 hours, from about 8 hours to about 12 hours, from about 12 hours to about 16 hours, from about 16 hours to about 24 hours, from about 24 hours to about 36 hours, from about 36 hours to about 48 hours, from about 48 hours to about 72 hours, or from about 72 hours to about 1 week preceding stem cell donation.

Pretreatment with an ALDH2 agonist is useful, for example, to expand the number of hematopoietic cells in vivo, such that the population of stem cells in the individual available for stem cell donation is increased. The above situation is only one example of a circumstance when a subject would benefit from pretreatment with a suitable ALDH2 agonist. In another example, pretreatment with an ALDH2 agonist is useful to increase the potency of hematopoietic cells in vivo, such that the potency of stem cells in the individual available for stem cell donation is increased.

As described above in the section entitled “Methods of Protecting, Expanding and Increasing Potency of Hematopoietic Cells”, the present disclosure provides methods that optionally include introducing a treated population of hematopoietic cells (e.g., where the treatment is effected by contacting the cells with an ALDH2 agonist, e.g., an activator of ALDH2) into a recipient individual. Introduction of the treated hematopoietic cells is useful in a variety of applications.

In certain aspects, the present disclosure provides treatment regimens that combine the post-radiation therapy or post-chemotherapy treatment with the introduction of a treated population of hematopoietic cells (e.g., as described above) with the pre- and/or post-radiotherapy/chemotherapy administration (e.g., systemic and/or local administration) of an ALDH2 agonist to an individual (e.g., as also described above). As such, the present disclosure provides a treatment regimen wherein an individual with a cancer receives an administration of an ALDH2 agonist (e.g., an activator of ALDH2) before radiotherapy or chemotherapy, the treatment regimen further including introducing into the individual a treated population of hematopoietic cells as described above. The present disclosure further provides a treatment regimen wherein an individual with a cancer receives an administration of an ALDH2 agonist (e.g., an activator of ALDH2) and an administration of a treated population of hematopoietic cells, with both administrations occurring after the radiotherapy or chemotherapy. As will be appreciated, the present disclosure also provides a treatment regimen in which an ALDH2 agonist is administered systemically and/or locally to an individual before and after radiotherapy or chemotherapy, the treatment regimen further including the introduction of a treated population of hematopoietic cells after the radiotherapy or chemotherapy.

In certain aspects, the present disclosure provides a treatment regimen wherein an individual who has been exposed to one or more damaging toxins, receives an administration of an ALDH2 agonist (e.g., an activator of ALDH2) and an administration of a treated population of hematopoietic cells. As will be appreciated, the present disclosure also provides a treatment regimen in which an ALDH2 agonist is administered systemically and/or locally to an individual, wherein the treatment regimen further includes the introduction of a treated population of hematopoietic cells.

In certain aspects, the present disclosure further provides a treatment regimen wherein an individual with a genetic disease that leads to HSC damage, bone marrow failure, an autoimmune disease, or development of hematologic malignancies, receives an administration of an ALDH2 agonist (e.g., an activator of ALDH2) and an administration of a treated population of hematopoietic cells. As will be appreciated, the present disclosure also provides a treatment regimen in which an ALDH2 agonist is administered systemically and/or locally to an individual, wherein the treatment regimen further includes the introduction of a treated population of hematopoietic cells.

In some embodiments, subjects to be treated are humans. In some embodiments, a human to be treated according to a subject method is one that has two “wild-type” ALDH2 alleles, e.g., the ALDH2 encoded by the two wild-type ALDH2 alleles has a glutamic acid at position 487. In some embodiments, the individual to be treated has a hypomorphic mutation in ALDH2. In some cases the hypomorphic mutation is ALDH2*2.

In other embodiments, a human to be treated according to a subject method is one that has one or two “ALDH2*2” alleles, e.g., the ALDH2 encoded by one or both ALDH2 alleles comprises a lysine as amino acid position 487. See US 2011/0105602 for details of the amino acid sequence. The E487K polymorphism is a semidominant polymorphism, and results in an ALDH2 tetramer that has significantly lower enzymatic activity than “wild-type” ALDH2. Thus, subjects who are heterozygous or homozygous for the ALDH2*2 allele have much lower in vivo ALDH2 activity levels than subjects who are homozygous for the “wild-type” ALDH2 allele. Subjects who are heterozygous or homozygous for the ALDH2*2 allele are expected to benefit from treatment with a compound of the present disclosure, because the level of ALDH2 activity in such subjects is particularly low, and any increase of ALDH2 activity levels would be expected to provide a therapeutic effect. Any increase in ALDH2 activity would be beneficial in treating conditions such as ischemic disorders, in increasing the responsiveness of such subjects to nitroglycerin, etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Interferon Stress in HSCs in Response to Increased Aldehydic Load

DNA interstrand cross-links (ICL), which are normally repaired by the Fanconi Anemia (FA) complex gene products, can be induced by reactive aldehydes, e.g., acetaldehyde. A hypomorphic missense mutation of Aldehyde Dehydrogenase 2 (ALDH2) found in ˜560 million people worldwide results in decreased ability to metabolize acetaldehyde and other toxic aldehydes. The ALDH2*2 genotype causes the well-known disulfiram-like “Asian flushing syndrome” observed after ethanol ingestion, but also increased susceptibility to cancer, higher risk of aplastic anemia and more rapid progression to marrow failure in children with FA.

The effects of increased aldehydic load in HSC, resulting from genetic variation (ALDH2 *2) and environmental exposure (ethanol challenge to increase aldehydic load) were studied. Hematopoietic stem and progenitor cells (HSPCs) from wild type (WT) and ALDH2*2/*2 mice were serially examined between 6-30 weeks of age by immunophenotype, cell dynamics, competitive repopulation and gene expression analyses. 10 week old mice were challenged with ethanol or saline control for 8 weeks and analyzed.

It was observed that unchallenged ALDH2*2/*2 mice had progressive declines in long-term (LT) and short-term (ST) HSC numbers, and reduced numbers of mature blood cells. There was a 4-fold reduction in repopulating capacity of ALDH2*2/*2 HSC. Gene expression of ALDH2*2 HSCs showed signatures of interferon responses. Increased aldehydic load resulting from chronic ethanol exposure induced HSPC damage via increased apoptosis and proliferation.

In summary, these results indicated the common ALDH2*2 mutation, even without increased aldehydic load, caused HSC stress by increased apoptosis and decreased survival and self-renewal. This was exacerbated by aldehydic loading.

A series of small molecule activators were developed, which significantly increase the enzymatic activity of both wild type (WT) ALDH2 (ALDH2*1) and ALDH2*2 proteins. Without being bound to any particular theory, it is thought that decreasing the load of ICL-inducing reactive aldehydes by small molecule activators of ALDH2 (both WT ALDH2*1 and mutant ALDH2*2) could prevent or delay marrow failure, leukemia and other cancers in FA patients. Normalization of HSC dynamics and interferon signatures along with direct measure of aldehydic load, may be useful surrogate endpoints in clinical trials aimed at decreasing the aldehydic load to prevent aplastic anemia and leukemia in FA.

### Example 2

### ALDH2 Activator Protects and Expands HSCs In Vivo

Mice (wild-type C57BL/6N were administered with Alda-1 (10 mg/kg.day) or vehicle control (50% DMSO/50% PEG-400) by continuous infusion via subcutaneous osmatic pumps for 3 months.

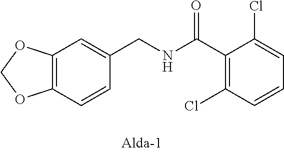 [SMILES: O=C(NCc1ccc2c(c1)OCO2)c1c(Cl)cccc1Cl]

Vehicle control and Alda-1 treated mice were then analyzed for HSC functionality. The mice treated with Alda-1 demonstrated significant HSC expansion. It was observed that mice treated with Alda-1 exhibited a 4-fold increase in (LT)-HSCs (FIG. 1A) and mice treated with Alda-1 exhibited a 2-fold increase in (ST)-HSCs (FIG. 1B). With reference to FIG. 1A and FIG. 1B, error bars show standard deviation, significance computed with Student's t-test. *=p≤0.05. In summary, chronic Alda-1 administration was well tolerated in vivo and resulted in expansion of HSC.

### Example 3

### ALDH2 Activator Increases the Repopulating Potential of HSCs

To measure the effects of ALDH2 activation on HSC potency, recipient mice were transplanted with purified HSCs from two donors, one of which had received Alda-1 prior to collection of HSCs for transplant, and another of the same strain which had not received Alda-1 prior to collection for transplant. The two donors were genetically the same except for a difference in the CD45 gene, which allows the blood cells from each to be distinguished from each other and the recipient.

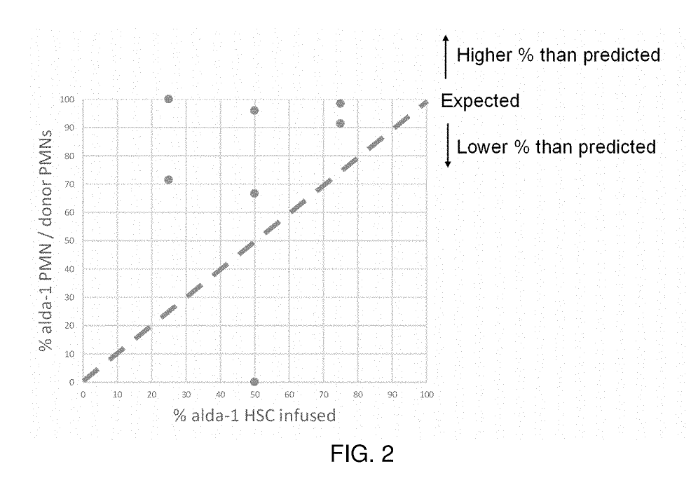

The Alda-1 treated donor had received Alda-1 subcutaneously for 2 months prior to HSC collection. The cells from the treated and untreated donors were mixed together prior to transplant at fixed ratios of 1:3 (25%), 1:1 (50%), or 3:1 (75%). A total of 3000 HSCs were given intravenously to immunodeficient (γc^{-/-} Kit^{W41/W41}) mice after low dose irradiation. The polymorphonuclear neutrophils (PMN, aka granulocytes) in each recipient mouse were tested at 6 weeks after transplant to determine what proportion of the PMN were derived from each HSC donor. As shown in FIG. 2, it was observed that PMN were more likely to be derived from the Alda-1 treated donor than predicted from the ratio of transplanted cells (diagonal linear line). This experiment demonstrated that Alda-1 treatment increased the potency of HSC in repopulating recipient.

These results are illustrated in FIG. 2, which demonstrates that competitive repopulation shows increased repopulating ability of HSC from Alda-1 treated mice. With reference to FIG. 2, the X-axis represents percentage of total cells infused which were from Alda-1 treated donor and the Y-axis represents the actual percentage of PMN observed after HSCT to be derived from Alda-1 treated donor. The linear line represents the predicted ratio if there were no difference in the repopulating ability of the HSC cells.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

Notwithstanding the appended claims, the disclosure set forth herein is also described by the following clauses.
Clause 1. A method of expanding hematopoietic cells, the method comprising contacting a starting population of hematopoietic cells with a therapeutically effective amount of at least one ALDH2 agonist, wherein the contacted hematopoietic cells have an expanded number of hematopoietic cells compared to the starting population of hematopoietic cells.
Clause 2. The method of clause 1, wherein the hematopoietic cells comprise hematopoietic stem cells (HSCs).
Clause 3. The method of clause 1, wherein the hematopoietic cells comprise hematopoietic stem and progenitor cells (HSPCs).
Clause 4. The method of any one of clauses 1 to 3, wherein said contacting is in vivo, and wherein said contacting comprises administering an effective amount of said at least one ALDH2 agonist to an individual in need thereof.
Clause 5. The method of clause 4, wherein said individual is undergoing chemotherapy treatment for cancer, has undergone or is about to undergo radiation treatment for cancer, or has been exposed to one or more damaging toxins.
Clause 6. The method of clause 4, wherein said individual has a genetic disease that leads to HSC damage, bone marrow failure, an autoimmune disease, or development of hematologic malignancies.
Clause 7. The method of clause 4, wherein the individual is a stem cell transplant donor.
Clause 8. The method of any one of clauses 1 to 3, wherein said contacting is ex vivo, and wherein said contacting generates an expanded population of hematopoietic cells.
Clause 9. The method of clause 8, further comprising introducing the expanded population of hematopoietic cells into a recipient individual.
Clause 10. The method of clause 9, wherein the expanded hematopoietic cells are expanded from a starting population of hematopoietic cells obtained from the recipient individual (i.e. autologous transplant).
Clause 11. The method of clause 9, wherein the expanded population of hematopoietic cells are expanded from a starting population of hematopoietic cells obtained from an individual other than the recipient individual (i.e. allogenic transplant).
Clause 12. The method of any one of clauses 9 to 11, wherein the recipient individual is a human.
Clause 13. The method of any one of clauses 1 to 3, wherein said contacting is in vitro, and wherein said contacting generates an expanded population of hematopoietic cells that are being genetically modified by a virus, a plasmid or CRISPR mediated gene therapy, or genomic editing.
Clause 14. The method of any one of clauses 1 to 3, further comprising contacting said starting population of hematopoietic cells in vitro with an antibody that specifically recognizes a marker associated with hematopoietic cells.
Clause 15. The method of clause 14, wherein the marker is selected from CD34, CD90, c-Kit, CD133, CD38, and combinations thereof.
Clause 16. The method of any one of clauses 1 to 15, further comprising contacting the starting population of hematopoietic cells with at least one growth factor selected from the group consisting of, stem cell factor (SCF), flt3 ligand (FL), interleukin-3 (IL3) and interleukin-6 (IL6).
Clause 17. The method of any one of clauses 1 to 16, wherein the ALDH2 agonist is of the formula (I):

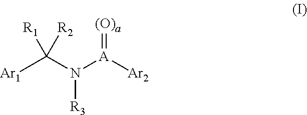

Wherein:

R₁, R₂, and R₃ are each independently selected from the group consisting of hydrogen, halogen, aryl, substituted aryl, alkyl, substituted alkyl, alkenyl, substituted alkenyl, alkynyl, substituted alkynyl, cycloalkyl, substituted cycloalkyl, heterocycle, substituted heterocycle, heteroaryl, substituted heteroaryl;

A is selected from C and S, wherein a is 1 when A is C and a is 2 when A is S; and

Ar₁ and Ar₂ are each independently selected from the group consisting of aryl, substituted aryl, heteroaryl and substituted heteroaryl,

or a pharmaceutically acceptable salt or solvate thereof.

Clause 18. The method any one of clauses 1 to 17, wherein the ALDH2 agonist is Alda-1:

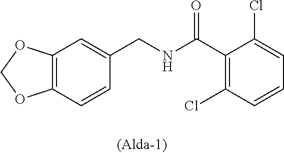 [SMILES: O=C(NCc1ccc2c(c1)OCO2)c1c(Cl)cccc1Cl]

or a pharmaceutically acceptable salt or solvate thereof.
Clause 19. The method of any one of clauses 1 to 18, wherein the hematopoietic cells are expanded by 2-fold or more.
Clause 20. The method of clause 19, wherein the hematopoietic cells are expanded by 4-fold or more.
Clause 21. The method of clause 4, wherein the individual has a hypomorphic mutation in ALDH2.
Clause 22. The method of clause 20, wherein the mutation is ALDH2*2.
Clause 23. A method of protecting hematopoietic cells, the method comprising contacting a starting population of hematopoietic cells with a therapeutically effective amount of at least one ALDH2 agonist, wherein the contacted hematopoietic cells are protected from damage caused by one or more of chemotherapy treatment, radiation treatment and exposure to one or more damaging toxins (e.g., as defined herein).
Clause 24. The method of clause 23, wherein the hematopoietic cells comprise hematopoietic stem cells (HSCs).
Clause 25. The method of clause 23, wherein the hematopoietic cells comprise hematopoietic stem and progenitor cells (HSPCs).
Clause 26. The method of any one of clauses 23 to 25, wherein said contacting is in vivo, and wherein said contacting comprises administering an effective amount of said at least one ALDH2 agonist to an individual in need thereof.
Clause 27. The method of clause 26, wherein said individual is undergoing chemotherapy treatment for cancer, has undergone or is about to undergo radiation treatment for cancer, or has been exposed to one or more damaging toxins.
Clause 28. The method of clause 26, wherein said individual has a genetic disease that leads to HSC damage, bone marrow failure, an autoimmune disease, or development of hematologic malignancies.
Clause 29. The method of clause 26, wherein the individual is a stem cell transplant donor.
Clause 30. The method of any one of clauses 23 to 25, wherein said contacting is ex vivo, and wherein said contacting generates a protected population of hematopoietic cells.
Clause 31. The method of clause 30, further comprising introducing the protected population of hematopoietic cells into a recipient individual.
Clause 32. The method of clause 30, wherein the protected hematopoietic cells are generated from a starting population of hematopoietic cells obtained from the recipient individual (i.e. autologous transplant).
Clause 33. The method of clause 30, wherein the protected population of hematopoietic cells are generated from a starting population of hematopoietic cells obtained from an individual other than the recipient individual (i.e. allogenic transplant).
Clause 34. The method of any one of clauses 30 to 33, wherein the recipient individual is a human.
Clause 35. The method of any one of clauses 23 to 25, wherein said contacting is in vitro, and wherein said contacting protects a population of hematopoietic cells that are being genetically modified by a virus, a plasmid or CRISPR mediated gene therapy, or genomic editing.
Clause 36. The method of any one of clauses 23 to 25, further comprising contacting said starting population of hematopoietic cells in vitro with an antibody that specifically recognizes a marker associated with hematopoietic cells.
Clause 37. The method of clause 36, wherein the marker is selected from CD34, CD90, c-Kit, CD133, CD38, and combinations thereof.
Clause 38. The method of any one of clauses 23 to 37, further comprising contacting the starting population of hematopoietic cells with at least one growth factor selected from the group consisting of, stem cell factor (SCF), flt3 ligand (FL), interleukin-3 (IL3) and interleukin-6 (IL6).
Clause 39. The method of any one of claims 23 to 38, wherein the ALDH2 agonist is of the formula (I):

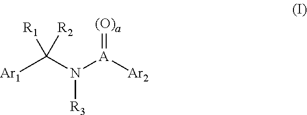

Wherein:

R₁, R₂, and R₃ are each independently selected from the group consisting of hydrogen, halogen, aryl, substituted aryl, alkyl, substituted alkyl, alkenyl, substituted alkenyl, alkynyl, substituted alkynyl, cycloalkyl, substituted cycloalkyl, heterocycle, substituted heterocycle, heteroaryl, substituted heteroaryl;

A is selected from C and S, wherein a is 1 when A is C and a is 2 when A is S; and

Ar₁ and Ar₂ are each independently selected from the group consisting of aryl, substituted aryl, heteroaryl and substituted heteroaryl,

or a pharmaceutically acceptable salt or solvate thereof.

Clause 40. The method any one of clauses 23 to 39, wherein the ALDH2 agonist is Alda-1:

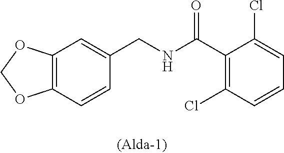 [SMILES: O=C(NCc1ccc2c(c1)OCO2)c1c(Cl)cccc1Cl]

or a pharmaceutically acceptable salt or solvate thereof.
Clause 41. The method of clause 26, wherein the individual has a hypomorphic mutation in ALDH2.
Clause 42. The method of clause 41, wherein the mutation is ALDH2*2.
Clause 43. A method of increasing the potency of hematopoietic cells, the method comprising contacting a starting population of hematopoietic cells with a therapeutically effective amount of at least one ALDH2 agonist, wherein the contacting increases the potency of the hematopoietic cells relative to the starting population of hematopoietic cells.
Clause 44. The method of clause 43, wherein the hematopoietic cells comprise hematopoietic stem cells (HSCs).
Clause 45. The method of clause 43, wherein the hematopoietic cells comprise hematopoietic stem and progenitor cells (HSPCs).
Clause 46. The method of any one of clauses 43 to 45, wherein said contacting is in vivo, and wherein said contacting comprises administering an effective amount of said at least one ALDH2 agonist to an individual in need thereof.
Clause 47. The method of clause 46, wherein said individual is undergoing chemotherapy treatment for cancer, has undergone or is about to undergo radiation treatment for cancer, or has been exposed to one or more damaging toxins.
Clause 48. The method of clause 46, wherein said individual has a genetic disease that leads to HSC damage, bone marrow failure, an autoimmune disease, or development of hematologic malignancies.
Clause 49. The method of clause 46, wherein the individual is a stem cell transplant donor.
Clause 50. The method of any one of clauses 43 to 45, wherein said contacting is ex vivo, and wherein said contacting generates a population of hematopoietic cells with increased potency relative to the starting population of hematopoietic cells.
Clause 51. The method of clause 50, further comprising introducing the population of hematopoietic cells with increased potency into a recipient individual.
Clause 52. The method of clause 51, wherein the hematopoietic cells with increased potency are generated from a starting population of hematopoietic cells obtained from the recipient individual (i.e. autologous transplant).
Clause 53. The method of clause 51, wherein the hematopoietic cells with increased potency are generated from a starting population of hematopoietic cells obtained from an individual other than the recipient individual (i.e. allogenic transplant).
Clause 54. The method of any one of clauses 51 to 53, wherein the recipient individual is a human.
Clause 55. The method of any one of clauses 43 to 45, wherein said contacting is in vitro, and wherein said contacting generates and increases the potency of a population of hematopoietic cells that are being genetically modified by a virus, a plasmid or CRISPR mediated gene therapy, or genomic editing.
Clause 56. The method of any one of clauses 43 to 45, further comprising contacting said starting population of hematopoietic cells in vitro with an antibody that specifically recognizes a marker associated with hematopoietic cells.
Clause 57. The method of clause 56, wherein the marker is selected from CD34, CD90, c-Kit, CD133, CD38, and combinations thereof.
Clause 58. The method of any one of clauses 43 to 57, further comprising contacting the starting population of hematopoietic cells with at least one growth factor selected from the group consisting of, stem cell factor (SCF), flt3 ligand (FL), interleukin-3 (IL3) and interleukin-6 (IL6).
Clause 59. The method of any one of clauses 43 to 58, wherein the ALDH2 agonist is of the formula (I):

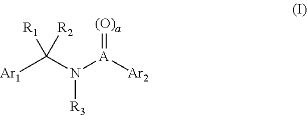

Wherein:

R₁, R₂, and R₃ are each independently selected from the group consisting of hydrogen, halogen, aryl, substituted aryl, alkyl, substituted alkyl, alkenyl, substituted alkenyl, alkynyl, substituted alkynyl, cycloalkyl, substituted cycloalkyl, heterocycle, substituted heterocycle, heteroaryl, substituted heteroaryl;

A is selected from C and S, wherein a is 1 when A is C and a is 2 when A is S; and

Ar₁ and Ar₂ are each independently selected from the group consisting of aryl, substituted aryl, heteroaryl and substituted heteroaryl,

or a pharmaceutically acceptable salt or solvate thereof.

Clause 60. The method of any one of clauses 43 to 59, wherein the ALDH2 agonist is Alda-1:

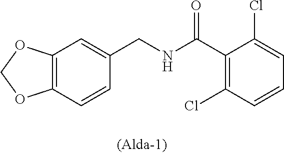 [SMILES: O=C(NCc1ccc2c(c1)OCO2)c1c(Cl)cccc1Cl]

or a pharmaceutically acceptable salt or solvate thereof.
Clause 61. The method of any one of clauses 43 to 60, wherein the potency of the hematopoietic cells is increased by 2-fold or more.
Clause 62. The method of clause 61, wherein the hematopoietic cells are expanded by 4-fold or more.
Clause 63. The method of clause 46, wherein the individual has a hypomorphic mutation in ALDH2.
Clause 64. The method of claim 63, wherein the mutation is ALDH2*2.

What is claimed is:

1. A method of treating hematopoietic cells, the method comprising: contacting a starting population of hematopoietic cells with a therapeutically effective amount of at least one ALDH2 agonist, wherein the contacting results in one or more of protecting, expanding and increasing the potency of the contacted hematopoietic cells relative to the starting population of hematopoietic cells.

2. The method of claim 1, wherein the hematopoietic cells comprise hematopoietic stem cells (HSCs).

3. The method of claim 1, wherein the hematopoietic cells comprise hematopoietic stem and progenitor cells (HSPCs).

4. The method of claim 1, wherein said contacting is in vivo, and wherein said contacting comprises administering an effective amount of said at least one ALDH2 agonist to an individual in need thereof.

5. The method of claim 4, wherein said individual is undergoing chemotherapy treatment for cancer, has undergone or is about to undergo radiation treatment for cancer, or has been exposed to one or more damaging toxins.

6. The method of claim 4, wherein said individual has a genetic disease that leads to HSC damage, bone marrow failure, an autoimmune disease, or development of hematologic malignancies.

7. The method of claim 4, wherein the individual is a stem cell transplant donor.

8. The method of claim 1, wherein said contacting is ex vivo, and wherein said contacting generates a treated population of hematopoietic cells.

9. The method of claim 8, further comprising introducing the treated population of hematopoietic cells into a recipient individual.

10. The method of claim 9, wherein the treated hematopoietic cells are generated from a starting population of hematopoietic cells obtained from the recipient individual (i.e. autologous transplant).

11. The method of claim 9, wherein the treated population of hematopoietic cells are generated from a starting population of hematopoietic cells obtained from an individual other than the recipient individual (i.e. allogenic transplant).

12. The method of claim 9, wherein the recipient individual is a human.

13. The method of claim 1, wherein said contacting is in vitro, and wherein said contacting generates a population of hematopoietic cells that are being genetically modified by a virus, a plasmid or CRISPR mediated gene therapy, or genomic editing.

14. The method of claim 1, further comprising contacting said starting population of hematopoietic cells in vitro with an antibody that specifically recognizes a marker associated with hematopoietic cells.

15. The method of claim 14, wherein the marker is selected from CD34, CD90, c-Kit, CD133, CD38, and combinations thereof.

16. The method of claim 1, further comprising contacting the starting population of hematopoietic cells with at least one growth factor selected from the group consisting of, stem cell factor (SCF), flt3 ligand (FL), interleukin-3 (IL3) and interleukin-6 (IL6).

17. The method of claim 1, wherein the ALDH2 agonist is of the formula (I): 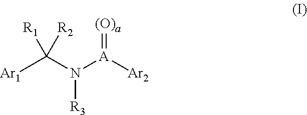
Wherein:
R₁, R₂, and R₃ are each independently selected from the group consisting of hydrogen, halogen, aryl, substituted aryl, alkyl, substituted alkyl, alkenyl, substituted alkenyl, alkynyl, substituted alkynyl, cycloalkyl, substituted cycloalkyl, heterocycle, substituted heterocycle, heteroaryl, substituted heteroaryl;
A is selected from C and S, wherein a is 1 when A is C and a is 2 when A is S; and
Ar₁ and Ar₂ are each independently selected from the group consisting of aryl, substituted aryl, heteroaryl and substituted heteroaryl, or a pharmaceutically acceptable salt or solvate thereof.

18. The method of claim 1, wherein the ALDH2 agonist is Alda-1: 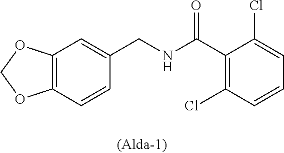 [SMILES: O=C(NCc1ccc2c(c1)OCO2)c1c(Cl)cccc1Cl]
or a pharmaceutically acceptable salt or solvate thereof.

19. The method of claim 1, wherein the hematopoietic cells are expanded by 2-fold or more.

20. The method of claim 19, wherein the hematopoietic cells are expanded by 4-fold or more.

21. The method of claim 1, wherein the potency of the hematopoietic cells is increased by 2-fold or more.

22. The method of claim 29, wherein the potency of the hematopoietic cells is increased by 4-fold or more.

23. The method of claim 4, wherein the individual has a hypomorphic mutation in ALDH2.

24. The method of claim 23, wherein the mutation is ALDH2*2.
